(19) United States
(12) Patent Application Publication
(10) Pub. No.: US 2017/0364630 A1
(43) Pub. Date: Dec. 21, 2017

(54) IDENTIFICATION OF CELLULAR ANTIMICROBIAL DRUG TABLETS THROUGH INTERACTOME ANALYSIS

(71) Applicant: Vanderbilt University, Nashville, TN (US)

(72) Inventors: Donald H. Rubin, Nashville, TN (US); Feixiong Cheng, Malden, MA (US); Zhongming Zhao, Nashville, TN (US)

(21) Appl. No.: 15/533,200

(22) PCT Filed: Dec. 7, 2015
(86) PCT No.: PCT/US15/64273

§ 371 Date: Jun. 5, 2017

Related U.S. Application Data
(60) Provisional application No. 62/087,979, filed on Dec. 5, 2014.

(51) Int. Cl. G06F 19/12; C12Q 1/70

(52) U.S. Cl. CPC G06F 19/12; C12Q 1/705; C12Q 1/701; C12Q 2600/178; C12Q 2600/136

(57) ABSTRACT

A method of identifying a promising cellular antiviral or bacterial toxin drug target is described including: 1) providing a plurality of potential antiviral or bacterial toxin drug targets; 2) generating an interactome including the potential drug targets using a systems-biology computational method; and 3) analyzing the interactome to identify one or more promising antiviral or bacterial toxin drug targets. New indications for older drugs identified using this method are also described.

### CROSS-REFERENCE TO RELATED APPLICATIONS

This application claims priority from U.S. Provisional Application Ser. No. 62/087,979, filed Dec. 5, 2014, the entire contents of which is incorporated herein by reference.

### BACKGROUND

Infectious diseases result in millions of deaths and cost billions of dollars annually. As of 2012, 35.3 million people worldwide were living with human immunodeficiency virus (HIV), and an estimated 1.6 million acquired immunodeficiency syndrome (AIDS)-related deaths were reported in 2012. In March 2014, the Worth Health Organization reported a major Ebola virus outbreak in the western African nation of Guinea. As of Mar. 25, 2015, over 26,000 suspected Ebola-infected cases had been identified, with over 10,000 deaths, and these numbers may be vastly underestimated. Infections by the Ebola and Marburg filoviruses cause a rapidly fatal hemorrhagic fever in humans for which no approved antiviral agents are available Reversion of advanced Ebola virus disease in nonhuman primates with ZMapp. Traditional antiviral drug discovery pipelines have yielded notable successes in recent years. However, two factors continue to provide commercial and medical incentives for developing more innovative and effective antiviral therapeutics, namely the propensity of viruses to develop drug resistance and the side effects caused by antiviral agents.

Therapeutic drugs often have more than one indication, which means that there is more than one particular disease for which it is used. The Food and Drug Administration (FDA) classifies indications for drugs in the United States. Indications for drugs can be classified in two categories: (1) FDA-approved, also called labeled indications, and (2) Non FDA-approved, also called off-label indications. With faster development times, increased safety, and decreased pharmacokinetic uncertainty, the prospect of drug repositioning (finding new indications for existing FDA-approved drugs) is emerging as a promising alternative to traditional drug design and offers an improved risk-benefit trade-off in combating infectious diseases (Taylor C M, Martin J, Rao R U, Powell K, Abubucker S, et al. (2013) PLoS Pathog 9: e1003149; and Cheng F, Liu C, Jiang J, Lu W, Li W, et al. (2012) PLoS Comput Biol 8: e1002503).

An interactome is the whole set of molecular interactions in a particular cell. The term specifically refers to physical interactions among molecules (such as those among proteins, also known as protein-protein interactions or PPIs) but can also describe sets of indirect interactions among genes. Viruses require host cellular factors for successful replication. Viral interactomes are connected to their host interactomes, forming virus-host interaction networks. Therefore, a comprehensive systems-level investigation of the molecular interactions between a virus and a host gene (i.e., a virus-host interactome) is crucial for understanding the roles of host factors with the end goal of discovering new druggable antiviral targets. (Chasman et al. (2014) PLoS Comput Biol 10: e1003626). In this regard, quantitative temporal viromics (Weekes et al. (2014) Cell 157: 1460-1472) and viral open reading frames (Pichlmair et al. (2012) Nature 487: 486-490) can be useful in studying the virus-host interactome (Jager et al. (2012) Nature 481: 365-370). However, the incorrect assignment of biological activities to viral and host factors, and the limited scale of experimental techniques have limited these approaches (Peng et al. (2009) Curr Opin Microbiol 12: 432-438).

### SUMMARY OF THE INVENTION

Using libraries of randomly mutagenized cells and gene-trap insertional analysis coupled with known drug-gene signatures, bioinformatics and network analysis, the inventors have discovered host cellular genes that are essential for the replication of a number of cytotoxic mammalian viruses and bacterial toxins. In addition, host antiviral and bacterial toxin gene targets were identified that are likely to be inhibited using known existing drugs with good pharmacokinetics profiles, allowing for the development of new broadly active antimicrobial therapeutics.

In view of these discoveries, the present invention provides a method of identifying a promising cellular antiviral or bacterial toxin drug target. The method includes: (1) providing a plurality of potential antiviral or bacterial toxin drug targets; (2) generating an interactome including the potential drug targets using a systems-biology computational method; and (3) analyzing the interactome to identify one or more promising antiviral or bacterial toxin drug targets.

In another aspect, the present invention provides a method of identifying a new antiviral drug indication. The method includes: (1) providing a plurality of potential antiviral drug targets; (2) generating an interactome including the potential antiviral drug targets using a systems-biology computational method; (3) analyzing the interactome to identify one or more promising antiviral drug targets; and (4) comparing a list of antiviral drug-gene signatures with the one or more promising antiviral drug targets to identify a new antiviral drug indication.

In another aspect of the invention, a method of treating a subject having an HIV-1 infection is provided. The method includes administering to the subject a therapeutically effective amount of a compound selected from the group consisting of alsterpaullone, lycorine, sanguinarine, testosterone, amylocaine, 2,6-dimethylpiperidine, triprolidine, fursultiamine, trichostatin A, and doxorubicin.

In yet another aspect, a method of treating a subject having a RSV infection is provided. The method includes administering to the subject a therapeutically effective amount of a compound selected from the group consisting of etamsylate, nicardipine, disulfiram, scoulerine, midecamycin, tyrphostin AG-825, hydroxyachillin, decamethonium bromide, PNU-0293363, and propantheline bromide.

Another aspect of the invention provides a method of treating a subject having an HSV-2 infection. The method includes administering a therapeutically effective amount of a compound selected from the group consisting of meclofenoxate, nocodazole, ellipticine, nilutamide, thioridazine, calycanthine, PF-00562151-00, trichostatin A, valproic acid, and digitoxigenin.

In another aspect, a method of treating a subject having an Ebola virus infection is provided. The method includes administering a therapeutically effective amount of a compound selected from the group consisting of piroxicam, azlocillin, and staurosporine.

### BRIEF DESCRIPTION OF THE FIGURES

FIGS. 1A-1F provide a diagram of the integrative antimicrobial drug discovery pipeline. (A) The gene-trap insertional mutagenesis approach employs a moloney murine leukemia virus (MMLV)-based shuttle vector that randomly integrates into host cell chromosomes and contains a promoterless neomycin-resistance gene. Shuttle vector integration between a host-cell promoter and an early exon disrupts (traps) the gene, allowing neomycin selection and derivation of a gene-trap library. (B) Host genes mediating the toxic effects of lytic viral replication or exposure to toxins were identified by: (i) selecting gene-trap libraries in neomycin; (ii) exposing gene-trap library cells to a lytic virus or a toxin; (iii) isolating surviving clones; (iv) resistance confirmation in surviving clones following exposure to a 10-fold higher dose of the virus or toxin studied; and (v) identification of the trapped gene by digesting genomic DNA to liberate shuttle vectors, self-ligation, bacterial transform, ampicillin selection, and sequencing trapped genes in the recovered plasmids. (C) Construction of a virus-host interaction network, where nodes (squares) are viruses and host cell genes (circles) are colored based on the human protein subcellular location and edges (lines) denote interactions. (D) Construction of an antiviral drug-gene interaction network. (E) Bioinformatics and network analysis of the virus/toxin-host interactome. (F) Identification of candidates for antiviral drug repositioning approach by incorporating drug-gene signatures from the Connectivity Map into the global virus-host interactome.

FIGS. 2A-2E shows bioinformatics and network analysis of the virus-host interactome. (A) Classification of trapped genes into functional categories by Ingenuity Pathway Analysis. (B) Distribution of new discovered virus-host interaction pairs for 13 viruses, 1 bacteria and 5 toxins. (C) Reactome pathway enrichment analysis of four different host cellular gene sets identified by gene-trap insertional mutagenesis (trapped genes), previous RNA interference (RNAi) screening studies, viral open reading frames (viORFs), and co-immunoprecipitation and liquid chromatography-mass spectrometry (Co-IP+LC/MS). (D) Global pathogen-host interaction network identified by the genome-wide gene-trap insertional mutagenesis approach, where toxins and bacteria are represented by red and cyan squares respectively. (E) Global virus-host interaction network identified by the genome-wide gene-trap insertional mutagenesis approach, where nodes (squares) are viruses, host cell gene products (circles) are colored based on their subcellular locations, and edges (lines) denote interactions.

FIG. 3 shows network topological and evolutionary characteristics of host genes mediating viral replication. Venn diagram showing the relationship between 859 host genes (trapped genes) identified by gene-trap insertional mutagenesis and (A) innate immunity genes and (B) human essential genes. (C) Boxplots showing the connectivity distribution of virus host genes (red) versus non-virus-host genes (light blue) in the physical protein interaction network (PIN) and large-scale computationally predicted protein interaction network (CPIN). (D) Evolutionary characteristics of virus-host genes (red) versus non-virus-host genes (light blue). (E) Node connectivity distribution of host genes identified by gene-trap insertional mutagenesis and three published gene sets: previous RNA interference (RNAi) screening studies, viral open reading frames (viORFs), and co-immunoprecipitation and liquid chromatography-mass spectrometry (Co-IP+LC/MS) and all proteins (Whole) in the PIN. (F) Gene dN/dS ratio cumulative distribution for four different gene sets and whole human genome (Whole). Mya: million years ago. P values in A and B were calculated using Fisher's exact test. P values in C and D were calculated using Wilcoxon rank-sum test.

FIG. 4 shows a human cell cycle phase-specific virus-host gene network. (A) Human cell cycle phase-specific virus-host gene network for host genes identify by gene-trap insertional mutagenesis. The concise overview of cell cycle regulation for gene MYC (B) and TAF4 (C). Dark color represents high expression across different cell cycle phases. B and C were prepared by Cyclebase 3.0.

FIG. 5 illustrates disease etiology analysis of virus target genes. (A) Venn diagram denoting the overlap among virus-target genes (Host genes), the catalogue of cancer genes (CCG), Mendelian disease genes (MDG), and orphan-disease mutated genes (ODMG). (B) Venn diagram denoting the overlap among virus-target genes (Host genes), genes whose mutations are significantly associated with cancer (Driver), genes in the Cancer Gene Census (CGC, experimentally validated cancer genes), and essential genes (Essential). (C) Disease gene enrichment analysis of virus-target genes (solid bars) versus nonvirus-target genes (striped bars). P values are calculated using Fisher's exact test.

FIG. 6 shows novel viral perturbations of the innate immunity network revealing new cancer etiologies. In this network, nodes represent viruses (squares), cancer types (hexagons), and genes (circles). Edges represent virus-host interactions (solid red arrows), cancer-gene associations (striped red arrows), and innate immunity protein-protein interactions (solid gray lines). Various cancer types represented are abbreviated as follows: breast invasive carcinoma (BRCA), bladder urothelial carcinoma (BLCA), colon adenocarcinoma (COAD), diffuse large B-cell lymphoma (DLBCL), glioblastoma multiforme (GBM), head and neck squamous cell carcinoma (HNSC), acute myeloid leukemia (LAML), kidney renal clear cell carcinoma (KIRC), lung adenocarcinoma (LUAD), multiple myeloma (MM), and uterine corpus endometrial carcinoma (UCEC).

FIG. 7 shows a global antiviral bipartite drug-target interaction network. In this network, nodes represent 691 virus-target genes (Host genes, squares) or known drugs (2,071) shown in circles, and where edges denote the interactions. Host gene products were colored based on their known subcellular locations, as shown in FIG. 2. All drugs were grouped using the anatomical therapeutic chemical (First-level ATC code) classification system.

FIG. 8 shows a drug-target interaction network for inhibiting anti-Ebola virus replication. (A) Newly discovered anti-Ebola virus drug-target interaction network, where nodes represent drugs (hexagons) or host genes (circles), and edges represent up-regulated (red lines) or down-regulated (blue lines) genes following drug treatment, as determined using the Connectivity Map data. Target gene product nodes were colored based on their subcellular locations, and drug nodes were colored based on P values (Fisher's exact test) calculated by our proposed computational approach (FIG. 1F). (B) Chemical structures of three selected drugs with significant P values.

### DETAILED DESCRIPTION OF THE INVENTION

The present invention provides methods for the identifying a promising candidate cellular antiviral or bacterial toxin drug target and for the treatment of viral infections. The present invention is based, in part on the discovery of an effective strategy for identifying promising druggable antiviral and bacterial toxin host gene targets by merging genome-wide gene-trap insertional mutagenesis, drug-gene network, and bioinformatics data. In addition, the present invention is further related to the use of a computable representation of genetic testing to effectively identify new potential antiviral and antibacterial indications for existing drugs.

### Definitions

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

The terms “comprising” and variations thereof do not have a limiting meaning where these terms appear in the description and claims.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc.

As utilized herein, by “prevent,” “preventing,” or “prevention” is meant a method of precluding, delaying, averting, obviating, forestalling; stopping, or hindering the onset, incidence, severity, or recurrence of intoxication and/or infection. For example, the disclosed method is considered to be a prevention if there is about a 10% reduction in onset, incidence, severity, or recurrence of intoxication and/or infection, or symptoms of intoxication and/or infection (e.g., inflammation, fever, lesions, weight loss, etc.) in a subject exposed to a toxin and/or a pathogen when compared to control subjects exposed to a toxin and/or a pathogen that did not receive a composition for decreasing intoxication and/or infection. Thus, the reduction in onset, incidence, severity, or recurrence of infection can be about a 10, 20, 30, 40, 50, 60, 70, 80, 90, 100%, or any amount of reduction in between as compared to control subjects. For example, and not to be limiting, if about 10% of the subjects in a population do not become intoxicated and/or infected as compared to subjects that did not receive preventive treatment, this is considered prevention.

“Pharmaceutically acceptable” as used herein means that the compound or composition is suitable for administration to a subject to achieve the treatments described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect, such as the up or down regulation of an identified host gene target by a detectable amount.

The term “subject” for purposes of treatment includes any human or animal subject who is infected by a virus, bacteria or related toxin. For methods of prevention the subject is any human or animal subject, and preferably is a human subject who is at risk of virus and/or bacterial infection. Besides being useful for human treatment, the identified therapeutic compounds of the present invention are also useful for veterinary treatment of mammals, including companion animals and farm animals, such as, but not limited to dogs, cats, horses, cows, sheep, and pigs. Preferably, subject means a human.

As used herein, the term “nucleic acid” refers to single or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the moieties discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphoroselenoate deoxynucleotides), or methyl groups (yielding methylphosphonate deoxynucleotides), a reduction in the AT content of AT rich regions, or replacement of non-preferred codon usage of the expression system to preferred codon usage of the expression system. The nucleic acid can be directly cloned into an appropriate vector, or if desired, can be modified to facilitate the subsequent cloning steps. Such modification steps are routine, an example of which is the addition of oligonucleotide linkers which contain restriction sites to the termini of the nucleic acid. General methods are set forth in in Sambrook et al. (2001) Molecular Cloning—A Laboratory Manual (3rd ed.) Vol. 1-3, Cold Spring Harbor Laboratory, Cold Spring Harbor Press, NY, (Sambrook).

### Identification of Cellular Antiviral and Bacterial Toxin Drug Targets

For many important functions, viruses either encode proteins closely related to host proteins or have evolved the ability to directly co-opt the services of cellular factors. Antiviral drugs are known to typically function by one of two broad strategies. Because viruses replicate by using self-encoded proteins or by seizing control of a host's cellular factors, drugs effective to interrupt viral replication can be identified that target either a viral or a cellular polypeptide drug target.

In addition, adhesion of bacteria to host surfaces is a crucial aspect of host colonization as it prevents the mechanical clearing of pathogens and confers a selective advantage towards bacteria of the endogenous flora. Thus, bacteria have evolved a very large arsenal of molecular strategies allowing them to target and adhere to host cells including a wide range of bacterial surface factors with adhesive properties. These adhesins recognize and employ various classes of host cellular molecules including transmembrane proteins such as integrins or cadherins. Some of these adhesins, after allowing the binding of bacteria to host cell surfaces, are also triggering the internalization of bacteria inside host cells. Therefore, antibacterial drugs can be identified that target a host's cellular molecules which enable bacterial infection. Moreover, bacterial pathogens produce a plethora of proteins known as “toxins” and “effectors” that target a variety of physiological host processes during the course of infection. Bacterial toxins and effector proteins offer bacterial pathogens an advantage over their host by modulating host cell function, killing host cells, or even preventing programmed cell death. Once the toxins/effector proteins reach the host cellular surface and gain access to the inside of the host cells, they must also traffic to the correct compartment to mediate their effects. Therefore, drugs can be identified that target a host's cellular molecules and the genes which encode it, that mediate the harmful effects of bacterial toxins.

Accordingly, the present invention provides a method of identifying a promising cellular antiviral or bacterial toxin drug target. The method includes the steps of: 1) providing a plurality of potential antiviral or bacterial toxin drug targets; 2) generating an interactome including the potential drug targets using a systems-biology computational method; and 3) analyzing the interactome to identify one or more promising antiviral or bacterial toxin drug targets.

Potential drug targets are host genes involved in bacterial toxicity and viral infection. Host cellular gene drug targets can be identified by numerous methods including but not limited to gene-trap insertional mutagenesis (trapped genes), previously reported RNA interference (RNAi) screening studies (e.g., those identified by Harvard small interfering (siRNA) screening protocols), viral open reading frames (viORFs), and co-immunoprecipitation and liquid chromatography-mass spectrometry (Co-IP+LC/MS).

In some embodiments, host genes involved in bacterial toxicity and viral infection, replication and/or growth suitable for use as drug targets can be identified using gene trap methods. Gene-trap insertional mutagenesis is a high-throughput forward genetics approach to randomly disrupt (trap) host genes and discover cellular genes that are essential for viral viral infection, replication, and/or growth replication, but nonessential for host cell survival. This approach is based on two important principles: (i) viral infection must be toxic to the chosen host cell line, and (ii) disrupting a gene critical for completing the viral life cycle confers survivability during subsequent viral selection, provided that the host cell can survive following reduced or abolished expression of the mutagenized gene.

These gene trap methods are set forth in the Examples as well as in U.S. Pat. No. 6,448,000 and U.S. Pat. No. 6,777,177. U.S. Pat. Nos. 6,448,000 and 6,777,177, which are incorporated herein in their entireties by this reference.

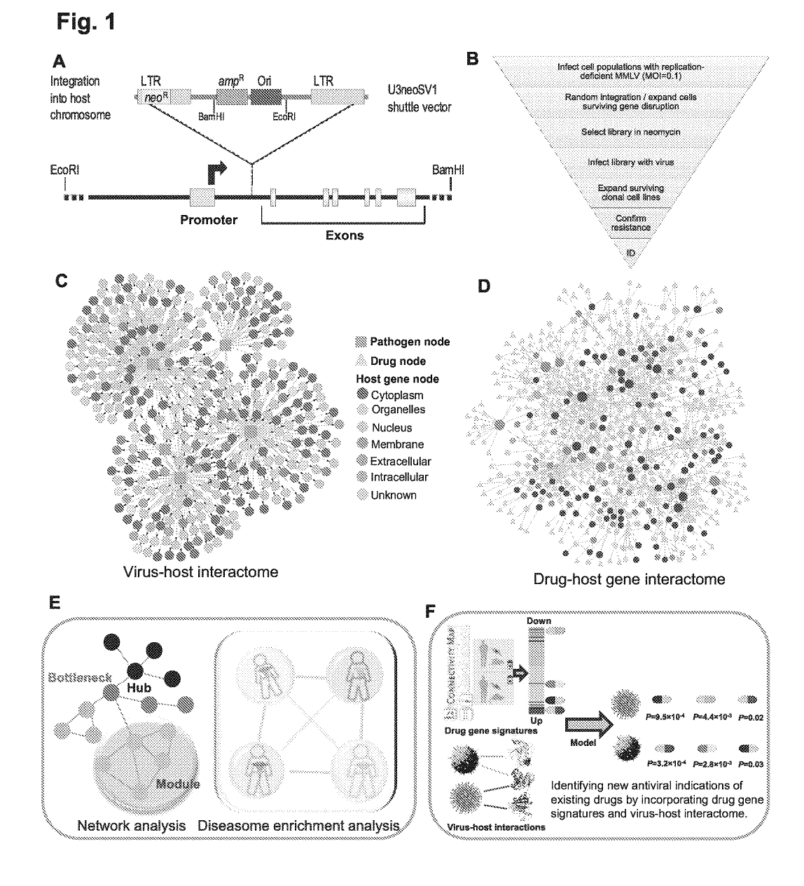

Briefly, genome-wide gene-trap insertional mutagenesis allows examination of the virus-host interactome (i.e., where viral interactomes are connected to their host interactomes, forming virus-host interaction networks), based on 6 steps: (i) random integration of an insertional mutagen shuttle vector containing a promoterless antibiotic-resistance gene (e.g., neomycin); (ii) antibiotic selection of cells expressing the antibiotic resistance gene; (iii) cytotoxic viral or bacterial toxin infection; (iv) resistance confirmation by re-infecting surviving clones at a 10-fold higher multiplicity of infection (MOI); (v) shuttle vector recovery from resistant clones (genomic DNA digestion, self-ligation, bacterial transformation, and ampicillin selection); and (vi) sequencing of trapped genes from bacterial colonies (see FIGS. 1A and 1B).

In exemplary embodiments, gene-trap insertional mutagenesis employs clonal gene-trap library cell lines including but not limited to Hep3B cells; MDCK cells; RIE-1 cells; TZM-b1 cells; and Vero E6 cells.

An insertional mutagen shuttle vector can include a viral vector. As used herein, “vector” means a cloning vector that contains the necessary regulatory sequences to allow transcription and translation of a cloned gene or genes. As used herein, the term “viral vector” refers to a vector that comprises a sequence that permits nucleic acid encoding a cloned nucleic acid sequence comprised by the vector to be incorporated into viral particles that are capable of delivering that sequence to a host cell by infection. It is understood in the art that some viral vector systems involve the use of helper virus or packaging cells that provide one or more functions not present on the viral vector comprising the cloned sequence to be delivered. Thus, a viral vector may encode all sequences necessary for viral particle assembly, or it can encode fewer than all such sequences, yet be part of a vector or cell system that directs the packaging of cloned sequence into infective viral particles. In some embodiments, the viral vector comprises an adenoviral vector. In another embodiment, the viral vector comprises a retroviral vector.

In an exemplary embodiment, the insertional mutagen shuttle vector includes a Moloney Murine Leukemia Virus-(MMLV) based retroviral vector sequence. In another exemplary embodiment, the insertional mutagen shuttle vector is a U3NeoSV1 promoter-trap provirus shuttle vector. The U3NeoSV1 promoter-trap provirus contains the ampicillin resistance (amp) gene and a plasmid origin of replication (Ori) flanked by the neomycin resistance (neo) gene in each long terminal repeat (LTR). Selecting for Neo resistant (NeoR) clones identifies those cells in which an endogenous gene has been disrupted as a result of the proviral insertion. Genomic DNA is isolated from mutagenized clones, digested with EcoRI, and then ligated and used to transform bacteria. Only bacteria that contain the vector will grow in the presence of ampicillin. Plasmid DNA is then prepared and sequenced to identify the gene mutated by the insertion of the promoter-trap vector.

As used herein, a gene “nonessential for cellular survival” means a gene for which disruption of one or both alleles results in a cell viable for at least a period of time which allows the toxicity of a toxin or viral replication to be decreased or inhibited in a cell. Such a decrease can be utilized for preventative or therapeutic uses or used in research. A gene necessary for bacterial toxicity or pathogenic infection or growth means the gene product of this gene, either protein or RNA, secreted or not, is necessary, either directly or indirectly in some way for the pathogen to grow. As utilized throughout, “gene product” is the RNA or protein resulting from the expression of a potential drug target gene.

The genes identified in a method in accordance with the present invention and their encoded proteins can be involved in any phase of the toxicity of a toxin or the viral life cycle including, but not limited to, toxin related cell membrane degradation, toxin related cell pore formation, toxin attachment to cellular receptors, toxin internalization, viral attachment to cellular receptors, viral infection, viral entry, internalization, disassembly of the virus, viral replication, genomic integration of viral sequences, transcription of viral RNA, translation of viral mRNA, transcription of cellular proteins, translation of cellular proteins, trafficking, proteolytic cleavage of viral proteins or cellular proteins, assembly of viral particles, budding, cell lysis and egress of virus from the cells.

Although the genes set forth herein are host cellular genes involved in toxin-induced toxicity or viral infection, as discussed throughout, the present invention is not limited to the specific genes listed as being involved in a bacterial toxicity and viral infection. Therefore, any of these host genes, or the proteins encoded by these host genes, can be involved in toxicity and infection by any infectious pathogen such as a fungus or a parasite which includes involvement in any phase of the infectious pathogen's life cycle.

As utilized herein, when referring to a potential drug target gene described herein, what is meant is any gene, any gene product, or any nucleic acid (DNA or RNA) associated with that gene name or a pseudonym thereof, as well as any protein, or any protein from any organism that retains at least one activity of the protein associated with the gene name or any pseudonym thereof which can function as a nucleic acid or protein utilized by a pathogen.

### Infective Viruses

Antiviral drug targets identified by the method of the invention can include a target suitable for treatment of viral infection by a virus selected from the group consisting of RNA viruses (including negative stranded RNA viruses, positive stranded RNA viruses, double stranded RNA viruses and retroviruses), or DNA viruses. All strains, types, and subtypes of RNA viruses and DNA viruses are contemplated herein.

In some embodiments, the antiviral drug target is a target suitable for treatment of viral infection by a virus selected from the group consisting of bovine viral diarrhea virus, cowpox virus, Dengue fever virus, Ebola virus, HIV-1, Herpes Simplex virus, Marburg virus, poliovirus, reovirus, rhinovirus 2, rhinovirus 16, and respiratory syncytial virus.

In some embodiments, potential drug targets can include drug targets suitable for decreasing toxicity by a bacterial toxin. A bacterial toxin drug target can include a target suitable for decreasing toxicity by a bacterial toxin selected from the group consisting of a Clostridium difficile toxin. More specifically, and not to be limiting the Clostridium toxin can be a Clostridium perfringens alpha toxin, Clostridium perfringens beta toxin, Clostridium perfringens epsilon toxin, Clostridium perfringens delta toxin, Clostridium perfringens theta toxin, Clostridium perfringens kappa toxin, Clostridium perfringens lambda toxin, Clostridium perfringens mu toxin, Clostridium perfringensnu toxin, Clostridium perfringens gamma toxin, Clostridium perfringens eta toxin, Clostridium difficile toxin A, Clostridium difficile toxin B, Clostridium botulinum A toxin, Clostridium botulinum B toxin, Clostridium botulinum C toxin, Clostridium botulinum D toxin, Clostridium botulinum E toxin, Clostridium botulinum F toxin or Clostridium botulinum G toxin.

### Bacterial Toxins

A bacterial toxin drug target can include a target suitable for decreasing toxicity by a bacterial toxin selected from the group consisting of a ricin toxin, saxitoxin, tetrodotoxin, abrin, conotoxin, E. coli toxin, streptococcal toxins, diphtheria toxin, cholera toxin, pertussis toxin, pseudomonas toxin, bacillus toxin, shigatoxin, T-2 toxin, anthrax toxin, cyanotoxin, hemotoxin, necrotoxin, or a mycotoxin, such as aflatoxin, amatoxin, citrinin, cytohalasin, ergotamine, fumonisin, gliotoxin, ibotenic acid, muscimol, ochratoxin, patulin, sterigmatocystin, trichothecene, vomitoxin, zeranol, and zearalenone.

In certain embodiments, the bacterial toxin drug target is a target suitable for decreasing toxicity by a bacterial toxin selected from the group consisting of Clostridium difficile TcdB toxin, C. perfringens α or β toxin, Helicobacter pylori vacuolating toxin, ricin toxin, and Staphylococcus aureus α toxin.

Systems-Biology Computation Methods

Genes identified that are part of a specific pathway or class can be identified as a potential druggable target. For example, cellular genes identified by gene-traps may be significantly enriched in innate immunity genes and human essential genes. In order to further evaluate the quality of host target genes identified by gene-trap insertional mutagenesis, several complementary systems biology-based analyses can be performed. Systems biology-based analyses can include, but are not limited to, gene-set enrichment analysis, pathway-enrichment analysis, protein interaction network topological analysis, and protein evolution analysis. Human protein interaction networks for use in the examining the topological network features can include one or more of a global physical protein interaction network (PIN), an atomic resolution three-dimensional structural protein interaction network (3DPIN), a kinase-substrate interaction network (KSIN), an innate immunity protein interaction network (INPIN), and a broad context computationally predicted protein interaction network (CPIN). To prevent data bias inherent to single-protein interaction networks, some embodiments include examining two or more independent human protein interaction networks. In order to investigate the biological function of the identified virus-target genes, topological network features can be examined, such as the degree of connectivity of virus-target gene products (proteins) in the human protein interactome.

Efficient selection of candidate drug targets can be significantly enriched in highly connected nodes, referred to as hubs, in human protein interaction networks. Network hubs can be defined as those nodes that ranked in the top twenty percent of the connectivity distribution. Nodes involved in the same biochemical process are highly interconnected. In certain embodiments, candidate virus-target proteins can represent innate immunity-related pathways or host gene products mediating viral replication, and the prevalence of these proteins has been found to be significantly enriched in hubs in several of the protein interaction networks examined. Accordingly, in one implementation, the search for candidate virus-target proteins can be focused on the hubs within these networks.

Network topology and pathway analysis can be performed using Cytoscape (version 2.8.3) and/or KEGG pathway analysis software. KEGG is a frequently-updated group of databases for the computerized knowledge representation of molecular interaction networks in metabolism, genetic information processing, environmental information processing, cellular processes and human diseases. The data objects in the KEGG databases are all represented as graphs and various computational methods for analyzing and manipulating these graphs are available. Cytoscape and PathwayAssist are similar software tools for automated analysis, integration and visualization of protein interaction maps. In these tools, automated methods for mining PubMed and other public literature databases are incorporated to facilitate the discovery of possible interactions or associations between genes or proteins. All of these resources may be useful in selecting pathways and nodes for pharmacological profiling according to our invention

Gene Set Enrichment Analysis (GSEA) is a computational method that determines whether an a priori defined set of genes shows statistically significant, concordant differences between two biological states (e.g., phenotypes) (also functional enrichment analysis) is a method to identify classes of genes or proteins that are over-represented in a large set of genes or proteins. The method uses statistical approaches to identify significantly enriched or depleted groups of genes, allowing for identification of common functions or pathways in a set of genes.

In one example, generating an interactome can include the systems-biology computational method of bioinformatics analysis. A cell cycle phase-specific sub-network can be implemented to systematically explore the cell cycle programming mechanisms for identified candidate viral-target genes. In some embodiments, identified candidate viral-target genes virus-target genes mediate cell cycle G0/1, S or G2 phases. In another example, which can be in addition to or in alternative to other methods of candidate target selection, generating an interactome can include the systems-biology computational method of diseasome enrichment analysis. For example, virus-target genes are significantly enriched in Mendelian disease genes, orphan disease-mutated genes, and somatic mutations in cancer genes, and selection of candidate virus-target genes can be focused accordingly.

In yet another example, which can be in addition to or in alternative to other methods of candidate target selection, generating an interactome can include the systems-biology computational method of evolutionary feature analysis. Evolutionary feature analysis can include examining the selective pressure and evolutionary rates of the virus-target genes identified. It has been determined that virus-target genes tend to undergo purifying selection, that is, the selective removal of alleles that are deleterious, in human evolutionary histories compared to non-virus target genes. In addition, the average time of divergence for virus-target gene products are significantly longer that of non-virus target gene products and the average evolutionary distance of virus-target gene products is also significantly higher than that observed for non-virus target gene products. Accordingly, selection of genes associated with viral replication tended to be ancient genes with low evolutionary rates compared to non-virus associated genes.

To identify new druggable targets, virus/bacterial toxin target genes identified by global RNAi screens and/or gene-trap insertional mutagenesis methods described above are cross-referenced with a drug-target database. Exemplary drug-target databases, include but are not limited to, DrugBank, the Therapeutics Target Database, and PharmGKB. Virus/Bacterial Toxin target genes included in one or more drug-target databases whose products can be targeted by approved drugs, investigational drugs, or pre-clinical agents, may then be referred to as a “druggable target gene”. In some embodiments, multiple candidate drugs can be identified for a given virus/bacterial toxin target gene. The selected drug can include known drugs, such as drugs already approved for the treatment of a human disease, and can be selected from a library of existing drugs. Selected drugs can include small compounds, peptides, proteins, ligands of cellular receptors, binding agents, such as antibodies or an antigen-binding fragments of antibodies, aptamers, adnectin, ribozyme, DNAzyme, or RNAi agents, such as, an siRNA, an shRNA, an antisense RNA, or a microRNA.

Once an interactome including the potential drug targets has been generated, the interactome is analyzed to identify one or more promising antiviral or bacterial toxin drug targets. The Connectivity Map (or CMap) is a reference catalog of gene-expression data collected from cultured human cells treated with chemical small molecule compounds and genetic reagents. The “Connectivity Map” resource together with pattern-matching software to mine these data can be used to find connections among small molecules sharing a mechanism of action, chemicals and physiological processes, and diseases and drugs.

In one implementation, new antiviral indications for existing drugs are determined computationally by incorporating drug-gene signatures from resources of the Connectivity Map into the global virus-host interactome. In this embodiment, if a given drug up- or down-regulates cellular genes to then inhibit the replication of a specific virus, then this drug may identified as an anti-infective agent. To determine this, an amplitude, a, for each drug-gene pair was extracted from CMap, the amplitude being defined as:

$$a = \frac{2 ( t - c )}{t + c} \qquad Eq . 1$$

where t is a scaled and thresholded average difference value for the drug treatment group in CMap, and c is a scaled and thresholded average difference value for the control group.

The amplitude values for each drug-gene pair can then be thresholded to categories each drug-gene pair as up-regulating, down-regulating, or non-regulating. In one implementation, pairs with amplitude values greater than 0.67 are categorized as up-regulating, pairs with amplitude values less than −0.67 are categorized as down-regulating, and pairs with amplitude values between −0.67 and 0.67 are categorized as non-regulating. In one analysis, around five hundred thousand drug-gene pairs were complied, connecting around one thousand three hundred drugs and around two thousand six hundred genes.

For each drug-virus pair, the number of host genes targeted by each virus and the number of genes that are up-regulated or down-regulated by each drug can be determined and used to form a contingency table. An appropriate multivariate analysis, such as Fisher's exact test, the G-test, Pearson's chi-squared test, or Bardard's test, can be applied to identify promising drugs for each virus. In one implementation, Fisher's exact test is used, with a correction for multiple comparisons, such as the Bonferroni correction or the Benjamini-Hochberg method. Drug-virus pairs having a corrected p-value from these analyses less than 0.1 were identified as significant.

### Therapeutic Methods Based on New Indications

Once a promising cellular antiviral or bacterial toxin drug target for a given virus/bacterial toxin target gene along with a known drug capable of up- or down-regulating the specific targeted cellular gene has been identified as described above, the drug may be administered to a subject for the treatment of a viral or bacterial toxin infection in a subject in need thereof.

In some embodiments, the methods described herein can be used to identify new antiviral drug indications for HIV-1 infection. By way of example, alsterpaullone, lycorine, sanguinarine, testosterone, amylocaine, 2,6-dimethylpiperidine, triprolidine, fursultiamine, trichostatin A, and doxorubicin were identified as having antiviral drug indications for HIV-1 infection in a human subject.

Therefore, another aspect of the invention provides a method of treating a subject having an HIV-1 infection by administering a therapeutically effective amount of a compound selected from the group consisting of alsterpaullone, lycorine, sanguinarine, testosterone, amylocaine, 2,6-dimethylpiperidine, triprolidine, fursultiamine, trichostatin A, and doxorubicin. In some embodiments, the compound is alsterpaullone, lycorine, or sanguinarine.

The methods described herein can be used to identify new antiviral drug indications for RSV infection. By way of example, etamsylate, nicardipine, disulfiram, scoulerine, midecamycin, tyrphostin AG-825, hydroxyachillin, decamethonium bromide, PNU-0293363, and propantheline bromide were identified as having antiviral drug indications for RSV infection in a human subject.

Therefore, another aspect of the invention provides a method of treating a subject having an RSV infection by administering a therapeutically effective amount of a compound selected from the group consisting of etamsylate, nicardipine, disulfiram, scoulerine, midecamycin, tyrphostin AG-825, hydroxyachillin, decamethonium bromide, PNU-0293363, and propantheline bromide.

A method of identifying a new antiviral drug indication described herein can be used to identify new antiviral drug indications for HSV-2 infection. By way of example, meclofenoxate, nocodazole, ellipticine, nilutamide, thioridazine, calycanthine, PF-00562151-00, trichostatin A, valproic acid, and digitoxigenin were identified as having antiviral drug indications for HSV-2 infection in a human subject.

Therefore, another aspect of the invention provides a method of treating a subject having an HSV2 infection by administering a therapeutically effective amount of a compound selected from the group consisting of meclofenoxate, nocodazole, ellipticine, nilutamide, thioridazine, calycanthine, PF-00562151-00, trichostatin A, valproic acid, and digitoxigenin.

The methods described herein can be used to identify new antiviral drug indications for Ebola virus infection. By way of example, piroxicam, azlocillin, and staurosporine were identified as having antiviral drug indications for Ebola virus infection in a human subject.

Therefore, another aspect of the invention provides a method of treating a subject having an Ebola virus infection by administering a therapeutically effective amount of a compound selected from the group consisting of piroxicam, azlocillin, and staurosporine.

Effective treatment can be any reduction from native levels and can be, but is not limited to, the complete ablation of the disease or the symptoms of the disease. Treatment can range from a positive change in a symptom or symptoms of intoxication and/or viral infection to complete amelioration of the bacterial intoxication and/or viral infection as detected by art-known techniques. For example, a disclosed method is considered to be a treatment if there is about a 10% reduction in one or more symptoms of the disease in a subject with the disease when compared to native levels in the same subject or control subjects. Thus, the reduction can be about a 10, 20, 30, 40, 50, 60, 70, 80, 90, 100%, or any amount of reduction in between as compared to native or control levels.

The therapeutic methods of the present invention can also result in a decrease in the amount of time that it normally takes to see improvement in a subject. For example, a decrease in infection can be a decrease of hours, a day, two days, three days, four days, five days, six days, seven days, eight days, nine days, ten days, eleven days, twelve days, thirteen days, fourteen days, fifteen days or any time in between that it takes to see improvement in the symptoms, viral load or any other parameter utilized to measure improvement in a subject. For example, if it normally takes 7 days to see improvement in a subject not taking the composition, and after administration of the composition, improvement is seen at 6 days, the composition is effective in decreasing infection. This example is not meant to be limiting, as one of skill in the art would know that the time for improvement will vary depending on the infection.

A pharmaceutical composition including an antiviral or bacterial toxin drug identified in accordance with a method described herein can be administered before or after intoxication and/or infection. The decrease in bacterial toxin toxicity in a subject need not be complete as this decrease can be a 10%, 20%, 30%, 40%, 50%, 60%, 70%, 80%, 90%, 100% or any other percentage decrease in between as long as a decrease occurs. This decrease can be correlated with amelioration of symptoms associated with toxicity and/or infection. These compositions can be administered to a subject alone or in combination with other therapeutic agents described herein, such as anti-viral compounds, antibacterial agents, antifungal agents, antiparasitic agents, anti-inflammatory agents, anti-cancer agents, etc. Examples of toxins, viral infections, and bacterial infections are set forth above. The compounds set forth herein or identified by the methods set forth herein can be administered to a subject to decrease toxicity and/or infection by any toxin, pathogen or infectious agent set forth herein.

### Administration and Formulation

Methods of introducing a pharmaceuticalcomposition described herein include, but are not limited to, mucosal, topical, intradermal, intrathecal, intranasal, intratracheal, via nebulizer, via inhalation, intramuscular, otic delivery (ear), eye delivery (for example, eye drops), intraperitoneal, vaginal, rectal, intravenous, subcutaneous, intranasal, and oral routes. Pharmaceutical compositions can be administered by any convenient route, for example by infusion or bolus injection, by absorption through epithelial or mucocutaneous linings (for example, oral mucosa, rectal, vaginal and intestinal mucosa, etc.) and can be administered together with other biologically active agents. Administration can be systemic or local. Pharmaceutical compositions can be delivered locally to the area in need of treatment, for example by topical application or local injection.

Pharmaceutical compositions are disclosed that include a therapeutically effective amount of a therapeutic agent, alone or with a pharmaceutically acceptable carrier. Furthermore, the pharmaceutical compositions or methods of treatment can be administered in combination with (such as before, during, or following) other therapeutic treatments, such as other antiviral agents, antibacterial agents, antifungal agents and antiparasitic agents.

For all of the therapeutic and administration methods disclosed herein, each method can optionally comprise the step of diagnosing a subject with an intoxication and/or infection or diagnosing a subject in need of prophylaxis or prevention of intoxication and/or infection.

The pharmaceutically acceptable carriers useful herein are conventional. Remington's Pharmaceutical Sciences, by Martin, Mack Publishing Co., Easton, Pa., 15th Edition (1975), describes compositions and formulations suitable for pharmaceutical delivery of the therapeutic agents herein disclosed. In general, the nature of the carrier will depend on the mode of administration being employed. For instance, parenteral formulations usually include injectable fluids that include pharmaceutically and physiologically acceptable fluids such as water, physiological saline, balanced salt solutions, aqueous dextrose, sesame oil, glycerol, ethanol, combinations thereof, or the like, as a vehicle. The carrier and composition can be sterile, and the formulation suits the mode of administration. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The composition can be a liquid solution, suspension, emulsion, tablet, pill, capsule, sustained release formulation, or powder. For solid compositions (for example powder, pill, tablet, or capsule forms), conventional non-toxic solid carriers can include, for example, pharmaceutical grades of mannitol, lactose, starch, sodium saccharine, cellulose, magnesium carbonate, or magnesium stearate. The composition can be formulated as a suppository, with traditional binders and carriers such as triglycerides.

Embodiments of the disclosure including medicaments can be prepared with conventional pharmaceutically acceptable carriers, adjuvants and counterions as would be known to those of skill in the art.

The amount of therapeutic agent effective in decreasing or inhibiting toxicity or infection can depend on the nature of the toxin or pathogen and its associated disorder or condition, and can be determined by standard clinical techniques. Therefore, these amounts will vary depending on the type of virus, bacteria, fungus, parasite or other pathogen. For example, the dosage can be anywhere from 0.01 mg/kg to 100 mg/kg. Multiple dosages can also be administered depending on the type of toxin or pathogen, and the subject's condition. In addition, in vitro assays can be employed to identify optimal dosage ranges. The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems.

In some embodiments, a therapeutically effective amount of an anti-bacterial toxin drug identified in accordance with a method described herein can include the amount required to decrease the toxicity of a bacterial toxin in a subject by either decreasing or increasing the expression or activity of at least one identified host gene or gene product identified as being involved in bacterial toxicity. Similarly, a therapeutically effective amount of an anti-viral drug identified in accordance with a method described herein can include the amount required to inhibit viral infection, replication and/or growth in a subject by either decreasing or increasing the expression or activity of at least one identified host gene or gene product identified as being involved in viral infection.

The disclosure also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

The present invention is illustrated by the following example. It is to be understood that the particular example, materials, amounts, and procedures are to be interpreted broadly in accordance with the scope and spirit of the invention as set forth herein.

### Example

### Systems Biology-Based Investigation of Cellular Antiviral Drug Targets Identified by Gene-Trap Insertional Mutagenesis

### Methods

### Cell Lines and Viruses

TZM-b1 cells were obtained from the NIH AIDS Research and Reference Reagent Program (Germantown, Md.). HepG2, Hep3B, L, MDCK, and Vero E6 cells were obtained from the American Type Culture Collection (ATCC; Manassas, Va.). Cowpox virus (Brighton strain), human rhinovirus 2 (HGP strain), human rhinovirus type 16 (11757 strain), influenza A virus (H1N1; A/PR/8/34 strain), poliovirus (Chat strain), and respiratory syncytial virus (A2 strain) were obtained from the ATCC. Dengue Fever Virus type 2 (16681 strain) was a generous gift from Dr. Guey Perng (Emory University). Herpes simplex virus type 1 (KA Strain) was kindly provided by Dr. David Knipe (Harvard University). Herpes simplex virus type 2 (186 strain) was a gift from Dr. Patricia Spear (Northwestern University). Reovirus type 1 (Lang strain) was obtained from Bernard N. Fields. Ebola virus (Zaire species, 1976 Mayinga strain) and Marburg virus (1967 Voege strain) were studied in a BSL4 containment facility at the Centers for Disease Control in Atlanta, Ga. The U3neoSV1 retrovirus shuttle vector (Hicks G G et al. (1997) Nature Genetics 16: 338-344) was used as an insertional mutagen to prepare gene-trap libraries with parental, virus-sensitive cells, as described (Murray J L, et al. (2005) J Virol 79: 11742-11751; Organ E L, et al. (2004) BMC Cell Biol 5: 41).

### Production of Clonal Gene-Trap Library Cell Lines Resistant to Lytic Viral Infection

Methods describing the preparation of clonal gene-trap library cell lines resisting lytic infection using Hep3B cells (Dengue fever virus); MDCK cells (influenza A); RIE-1 cells (reovirus); TZM-b1 cells (human rhinovirus 2 and 16); and Vero E6 cells (cowpox, Ebola, Herpes simplex virus 1 and 2, Marburg, poliovirus, and respiratory syncytial virus) were described previously (Murray J L, et al. (2005) J Virol 79: 11742-11751; Organ E L, et al. (2004) BMC Cell Biol 5: 41; Sheng J, et al. (2004) BMC Cell Biol 5: 32; Dziuba N, et al. (2012) AIDS Res Hum Retroviruses 28: 1329-1339; Murray J L, et al. (2012) Antivir Chem Chemother 22: 205-215; Murray J L, et al. (2014) Molecular Biotechnology 56: 429-437). Briefly, gene-trap libraries, each harboring approximately 10⁴ gene entrapment events, were expanded to 80-90% confluency until ˜10³ daughter cells represented each clone. The indicated cell lines were infected with a low MOI (range=0.0002-0.01), and infection proceeded until >90% cytopathic effects were observed (3-7 days). The medium was changed every 2-3 days until surviving clones were visible, which were generally observed after 2-3 weeks in culture. Surviving clones were expanded in duplicate wells of separate 24-well plates, and resistance was confirmed in clones by re-infecting 1 of the duplicate wells at a 10-fold higher MOI than the original cell populations were exposed to. Resistant clones showing >70% survival following re-infection were selected for expansion to identify trapped genes, using cells growing in the uninfected wells of 24-well plates.

Rescue and Sequencing the U3neoSV1 Shuttle Vector from Resistant Clones

Genomic DNA from clonal, virus-resistant cell lines was extracted using the QIAamp DNA Blood Mini Kit (Qiagen, Inc., Valencia, Calif.). Shuttle vectors and genomic DNA fragments flanking the U3neoSV1 integration site were recovered by digesting genomic DNA with either BamH1 or EcoRI, self-ligating the resulting genomic DNA fragments, transforming Escherichia coli, and selecting for bacteria harboring carbenicillin-resistant plasmids, as described (Organ E L, et al. (2004) BMC Cell Biol 5: 41). DNA sequences flanking the U3neoSV1 integration sites were sequenced using primers annealing to the U3neoSV1 shuttle vector.

### Construction of a High-Quality Human Protein Interactome

We downloaded protein-protein interaction data from various publications and bioinformatics databases. Because the current publicly available human protein interaction databases are still incomplete, we constructed 5 different yet complementary human PINs: (i) a large-scale physical PIN, (ii) a three-dimensional structural PIN, (iii) a kinase-substrate interaction network (KSIN), (iv) a comprehensive innate immunity PIN, and (v) a large-scale computationally predicted PIN, based on our previous studies (Cheng F, et al. (2014) Mol Biol Evol 31: 2156-2169; Cheng F, et al. (2014) Oncotarget 5: 3697-3710). We implemented 3 data cleaning steps. First, we defined high-quality interactions as those that have been experimentally validated in human models through a well-defined experimental protocol. Interactions that did not satisfy this criterion were discarded. Second, we annotated all protein-coding genes using gene Entrez ID, chromosome location, and the official gene symbols from the National Center for Biotechnology Information (NCBI) database (http://www.ncbi.nlm.nih.gov/), as described in detail previously (Cheng F, et al. (2014) Mol Biol Evol 31: 2156-2169; Cheng F, et al. (2014) Oncotarget 5: 3697-3710).

### Construction of the Drug-Gene Interactome

Drug-gene interactions (DGI) were acquired from the DrugBank database (v3.0) (Knox C, et al. (2011) Nucleic Acids Res 39: D1035-1041), the Therapeutic Target Database (TTD) (Zhu F, et al. (2012) Nucleic Acids Res 40: D1128-1136), and the PharmGKB database Hernandez-Boussard T, et al. (2008) Nucleic Acids Res 36: D913-918). Drugs were grouped using ATC classification system codes and annotated using Medical Subject Headings (MeSH) and Unified Medical Language System (UMLS) vocabularies (Bodenreider O (2004) Nucleic Acids Res 32: D267-270). All genes were mapped and annotated using the gene Entrez ID and official gene symbols found in the NCBI database. All duplicated DGI pairs were removed. In total, we obtained 17,490 DGI pairs connecting 4,059 FDA approved or investigational drugs and 2,746 gene products.

### Categories of Different Disease Gene Sets

Cancer driver genes. A set of 384 genes that are significantly mutated in cancer was selected from several large-scale cancer genomic analysis projects (Lawrence M S, et al. (2014) Nature 505: 495-501; Tamborero D, et al. (2013) Sci Rep 3: 2650; Vogelstein B, et al. (2013) Science 339: 1546-1558; Kandoth C, et al. (2013) Nature 502: 333-339).

Other cancer genes. Additional cancer genes were selected for bioinformatics analysis from the following resources. First, 487 experimentally validated cancer genes were downloaded on Jul. 10, 2013 from the Cancer Gene Census (Forbes S A, et al. (2011) Nucleic Acids Res 39: D945-950) and denoted as CGC genes. We also collected 4,050 cancer genes assembled in a previous study (Cheng F, et al. (2014) Mol Biol Evol 31: 2156-2169) referred to here as the comprehensive catalogue of cancer genes, CCG set. Together, these resources provide overlapping and complementary candidate cancer genes.

Mendelian disease genes (MDGs). A set of 2,714 MDGs was downloaded from the Online Mendelian Inheritance in Man (OMIM) database (Hamosh A, et al. (2005) Nucleic Acids Res 33: D514-517) in December 2012. The OMIM database contained 4,132 gene-disease association pairs connecting 2,716 disease genes in 3,294 Mendelian diseases or disorders.

Orphan disease-causing mutant genes (ODMGs). We collected 2,123 ODMGs from a previous study (Zhang M, et al. (2011) Am J Hum Genet 88: 755-766). The United States Rare Disease Act of 2002 defines a disease as an orphan disease that affects fewer than 200,000 individuals in the United States, the equivalent of approximately 6.5 people per 10,000 (Dear J W, et al. (2006) Br J Clin Pharmacol 62: 264-271).

Essential genes. Essential genes (2,719) were compiled from the Online Gene Essentiality (OGEE) Database (Chen W H, et al. (2012) Nucleic Acids Res 40: D901-906).

Cell cycle genes. Human host cell cycle genes (986 genes) regulating G0/1, S, and G2 phase transitions were collected from a previous study identified by a genome-wide RNAi screening (Kittler R, et al. (2007) Nat Cell Biol 9: 1401-1412).

Innate immune genes. Human innate immunity genes (971) playing a critical role in the innate immune response were collected from InnateDB Breuer K, et al. (2013) Nucleic Acids Res 41: D1228-1233).

### Computing Selective Pressure and Evolutionary Rates

We calculated dN/dS ratios (Hirsh A E, et al. (2005) Mol Biol Evol 22: 174-177) to examine selective pressures on genes. Initially, human-mouse orthologous genes were used to compute dN and dS substitution rates using human-mouse sequence data for 16,854 genes available in the Ensemble BioMart database. In addition, evolutionary rate ratios were determined, as described in a previous study (Bezginov A, et al. (2013) Mol Biol Evol 30: 332-346).

### Inferring Protein Evolutionary Origins

The evolutionary origin of a protein refers to the approximate date that the protein originated and can be inferred from phylogenetic analysis. We used the protein origin data from ProteinHistorian (Capra J A, et al. (2012) PLoS Comput Biol 8: e1002567). Specially, the origin (age) of a protein was estimated by considering 3 factors: the species tree, the protein family database, and the ancestral family reconstruction algorithm. Furthermore, evolutionary distances were calculated by comparing human sequences with orthologous sequences from other animals, as described (Bezginov A, et al. (2013)).

### Computational Identification of New Antiviral Indications for Existing Drugs

We collected drug-gene signatures from the Connectivity Map (CMap, build 02) (Lamb J, et al. (2006) Science 313: 1929-1935). The CMap is comprised of over 7,000 gene expression profiles from human cultured cell lines treated with various small bioactive molecules (1,309 total) at different concentrations, covering 6,100 individual instances. The CMap thus provides a measure of the extent of differential expression for a given probe set. The amplitude (a) was defined as follows:

$$a = \frac{t - c}{( t + c ) / 2}$$

where t is the scaled and thresholded average difference value for the drug treatment group and c is the thresholded average difference value for the control group. Thus, a =0 indicates no differential expression, a >0 indicates increased expression (up-regulation) upon treatment, and a<0 indicates decreased expression (down-regulation) upon treatment. For example, an amplitude of 0.67 represents a two-fold induction. Drug gene signatures with amplitudes of >0.67 were defined as up-regulated drug-gene pairs, and amplitudes <−0.67 reflected down-regulated drug-gene pairs. We then mapped probe sets into a global virus-host interactome. In total, we compiled ˜500,000 drug-gene pairs from the CMap connecting 1,309 drugs and 2,600 virus target genes.

For each drug-virus pair, we counted the number of host genes targeted by a given virus, those that are up- or down-regulated by drug treatments, as well as overlapping or mutually exclusive pairs (FIG. 1F). Next, we calculated P values by Fisher's exact test-corrected P values using Bonferroni's multiple comparison test in R package for each drug-virus pair. We then used q<0.1 as a cutoff to identify significant drug-virus pairs for antiviral drug repositioning.

### Network Topology Measurements

Network theory proposes that there are 2 important components of networks, namely nodes and edges. We studied virus-host bipartite networks, wherein nodes represented viruses and host cellular genes, and edges denoted interactions found by gene-trap insertional mutagenesis. For PIN studies, nodes were comprised of proteins and edges were based on known physical interactions, protein structure evidence, and phosphorylation. We calculated connectivity (degree) values using Cytoscape v3.0. Hubs were defined as nodes ranked in the top 20% in the connectivity distribution, based on two previous studies (Cheng F, et al. (2014) Mol Biol Evol 31: 2156-2169; Cheng F, et al. (2014) Oncotarget 5: 3697-3710).

### Functional Enrichment Analysis

We used ClueGO (Bindea G, et al. (2009) Bioinformatics 25: 1091-1093), a Cytoscape (v3.0.1) plug-in, and Ingenuity Pathway Analysis software, for enrichment analysis of genes in the Reactome or canonical KEGG pathways. A hypergeometric test was performed to estimate statistical significances, and all P values were adjusted for multiple testing using Bonferroni's correction (adjusted P values).

### Statistical Analysis and Network Visualization

All statistical tests were performed on the R-project for Statistical Computing platform (v3.01). All network visualization and related network topological parameters were presented using Cytoscape (v2.8.3).

### Results

### Developing a Global Virus-Host Interactome

An integrated antiviral drug-discovery approach was developed that involves gene-trap insertional mutagenesis, consolidated drug-gene signatures, and bioinformatics analysis to rank candidate antiviral targets and identify potential antiviral indications for existing drugs (FIG. 1). Genome-wide gene-trap insertional mutagenesis allows examination of the virus-host interactome, based on 6 steps: (i) random integration of an insertional mutagen shuttle vector containing a promoterless neomycin-resistance gene; (ii) neomycin selection of cells expressing neomycin aminotransferase; (iii) cytotoxic viral infection; (iv) resistance confirmation by re-infecting surviving clones at a 10-fold higher multiplicity of infection (MOI); (v) shuttle vector recovery from resistant clones (genomic DNA digestion, self-ligation, bacterial transformation, and ampicillin selection); and (vi) sequencing of trapped genes from bacterial colonies (FIGS. 1A and 1B). In this manner, we identified over 850 candidate host genes mediating the cytotoxic effects of 13 viruses (cowpox virus, Dengue fever virus (Dengue-2), Ebola virus, HIV-1, Herpes simplex viruses (HSV)-1 and HSV-2, influenza A virus (Influenza-A), Marburg virus, poliovirus (Polio), reovirus, rhinovirus-2 and -16, and respiratory syncytial virus (RSV)), of which 20% were identified in studies with multiple viruses in one or more cell types. Following the same general method for gene-trap studies outlined above, we also identified 97 host genes mediating the lytic effects of Francisella tularensis (tularensis) and 5 toxins (Clostridium difficile TcdB toxin, C. perfringens ε toxin, Helicobacter pylori vacuolating toxin, Staphylococcus aureus a toxin, and ricin toxin). Encouraged by these findings, we then developed a systems biology-based pipeline to characterize the candidate cellular antiviral targets through several complementary approaches, including network analysis, bioinformatics analysis, diseasome enrichment analysis, and evolutionary feature analysis (FIGS. 1C-1E). Finally, we computationally predicted several new antiviral indications for existing drugs by incorporating drug-gene signatures from the Connectivity Map (Lamb J, et al. (2006)) into the global virus-host interactome (FIG. 1F).

### Expanding the Known Virus-Host Interactome by Gene-Trap Insertional Mutagenesis

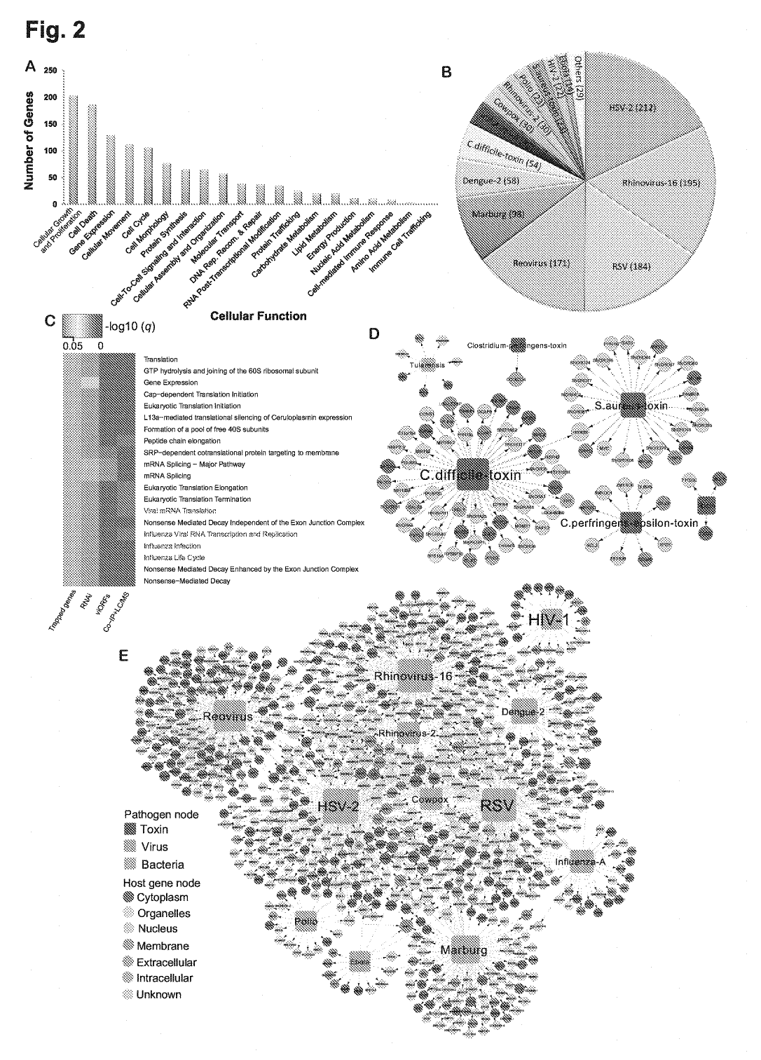

Our genome-wide gene-trap insertional mutagenesis studies revealed over 1,000 pathogen-host interactions that are essential for the replication of 13 cytotoxic mammalian viruses or the lytic effects of 1 bacteria and 5 toxins (FIGS. 2A and 2B). The curated gene set was further analyzed by Ingenuity Pathway Analysis software, which revealed their major biological roles, including cellular growth and proliferation, cell death, gene expression and protein synthesis, cell cycle regulation, cellular metabolic pathways, and other cellular functions (FIG. 2A). Using gene-trap insertional mutagenesis, we identified 1,179 new pathogen-host interactions connecting 859 host genes, 13 viruses, 1 bacteria, and 5 toxins (FIG. 2B). To gain further insight into their biological functions, we performed Reactome pathway-enrichment analysis for these 859 cellular host genes using ClueGO software [20]. As shown in FIG. 2C and Table 1, the most significantly enriched pathways associated with the gene set identified by gene-trap insertional mutagenesis included translation (adjusted p-value [q]=4.8×10⁻⁸, where q was based on a hypergeometric test p-value with Bonferroni correction), gene expression (q=9.9×10⁻⁸), mRNA splicing (q=3.6×10⁻⁷), viral mRNA translation (q=1.1×10⁻⁶), influenza viral RNA transcription and replication (q=6.9×10⁻⁶), and the influenza replication cycle (q=1.8×10⁻⁵).

TABLE 1
Top 20 significantly enriched Reactome pathways for 859 host genes identified by
gene-trap insertional mutagenesis.

| | | | Adjust P value |
|---|---|---|---|
| | | Term P | (Bonferroni |
| Pathway Source | GO Term | value | correction) |
| REACTOME_21.03.2014 | Translation | 2.10 × 10⁻¹⁰ | 4.80 × 10⁻⁸ |
| REACTOME_21.03.2014 | GTP hydrolysis and joining of | 2.09 × 10⁻¹⁰ | 4.82 × 10⁻⁸ |
| | the 60S ribosomal subunit | | |
| REACTOME_21.03.2014 | Gene Expression | 4.32 × 10⁻¹⁰ | 9.86 × 10⁻⁸ |
| REACTOME_21.03.2014 | Cap-dependent Translation | 5.71 × 10⁻¹⁰ | 1.30 × 10⁻⁷ |
| | Initiation | | |
| REACTOME_21.03.2014 | Eukaryotic Translation | 5.71 × 10⁻¹⁰ | 1.30 × 10⁻⁷ |
| | Initiation | | |
| REACTOME_21.03.2014 | L13a-mediated translational | 9.46 × 10⁻¹⁰ | 2.13 × 10⁻⁷ |
| | silencing of Ceruloplasmin | | |
| | expression | | |
| REACTOME_21.03.2014 | Formation of a pool of free 40S | 9.43 × 10⁻¹⁰ | 2.13 × 10⁻⁷ |
| | subunits | | |
| REACTOME_21.03.2014 | Peptide chain elongation | 1.04 × 10⁻⁹ | 2.34 × 10⁻⁷ |
| REACTOME_21.03.2014 | SRP-dependent cotranslational | 1.36 × 10⁻⁹ | 3.04 × 10⁻⁷ |
| | protein targeting to membrane | | |
| REACTOME_21.03.2014 | mRNA Splicing - Major | 1.63 × 10⁻⁹ | 3.61 × 10⁻⁷ |
| | Pathway | | |
| REACTOME_21.03.2014 | mRNA Splicing | 1.63 × 10⁻⁹ | 3.61 × 10⁻⁷ |
| REACTOME_21.03.2014 | Eukaryotic Translation | 3.30 × 10⁻⁹ | 7.29 × 10⁻⁷ |
| | Elongation | | |
| REACTOME_21.03.2014 | Eukaryotic Translation | 4.79 × 10⁻⁹ | 1.05 × 10⁻⁶ |
| | Termination | | |
| REACTOME_21.03.2014 | Viral mRNA Translation | 4.79 × 10⁻⁹ | 1.05 × 10⁻⁶ |
| REACTOME_21.03.2014 | Nonsense Mediated Decay | 1.00 × 10⁻⁸ | 2.19 × 10⁻⁶ |
| | Independent of the Exon | | |
| | Junction Complex | | |
| REACTOME_21.03.2014 | Influenza Viral RNA | 3.18 × 10⁻⁸ | 6.92 × 10⁻⁶ |
| | Transcription and Replication | | |
| REACTOME_21.03.2014 | Influenza Infection | 3.60 × 10⁻⁸ | 7.81 × 10⁻⁶ |
| REACTOME_21.03.2014 | Influenza Life Cycle | 8.11 × 10⁻⁸ | 1.75 × 10⁻⁵ |
| REACTOME_21.03.2014 | Nonsense Mediated Decay | 8.36 × 10⁻⁸ | 1.80 × 10⁻⁵ |
| | Enhanced by the Exon Junction | | |
| | Complex | | |
| REACTOME_21.03.2014 | Nonsense-Mediated Decay | 8.36 × 10⁻⁸ | 1.80 × 10⁻⁵ |

We next plotted the 1,179 new discovered pathogen-host interactions using two bipartite graphs: a toxin-host interaction network (FIG. 2D) and a virus-host interaction network (FIG. 2E), where nodes represent 859 host genes (circles), 13 viruses (orange squares), 1 Gram-negative bacteria (cyan square), and 5 toxins (red squares), and where edges represent interactions identified by gene-trap insertional mutagenesis. The host genes are grouped based on human protein subcellular locations, such as membranes, the cytoplasm, organelles, and the nucleus. To evaluate the quality of host genes identified by gene-trap insertional mutagenesis, we compared our network to three independent networks identified based on RNA interference (RNAi) screening studies, viral open reading frames (viORFs) (Pichlmair A, et al. (2012) Nature 487: 486-490), and co-immunoprecipitation and liquid chromatography-mass spectrometry (Co-IP+LC/MS) (Watanabe T, et al. (2014) Cell Host Microbe 16: 795-805). In total, we assembled 2,855 known virus-host interactions connecting 2,443 host genes and 55 pathogens identified from RNAi screens, 579 host proteins mediating 70 innate immune-modulating viORFs, and 1,292 host genes mediating influenza-host interactions identified by Co-IP+LC/MS, respectively. Several similar Reactome pathways, such as HIV infection (q=1.6×10⁻²⁰), HIV life cycle (q=1.7×10⁻²⁰), immune system (q=5.5×10⁻⁹), and mRNA splicing-minor pathway (q=3.2×10⁻⁷) were observed for the 2,443 host genes identified in previously reported RNAi screening studies (Table 2). However, several critical viral replication-related pathways, such as viral mRNA translation, influenza viral RNA transcription and replication, influenza infection, and influenza life cycle were significantly enriched among the host genes identified in gene-trap insertional mutagenesis, viROFs, and Co-IP+LC/MS studies, but not the RNAi gene set (FIG. 2C).

TABLE 2
Top 20 significantly enriched Reactome pathways for 2,443 host genes identified in
previously published RNAi screening studies.

| | | | Adjust P value |
|---|---|---|---|
| | | Term P | (Bonferroni |
| Pathway Source | GO Term | value | correction) |
| REACTOME_21.03.2014 | HIV Infection | 3.13 × 10⁻²³ | 1.57 × 10⁻²⁰ |
| REACTOME_21.03.2014 | HIV Life Cycle | 3.38 × 10⁻¹⁵ | 1.69 × 10⁻¹² |
| REACTOME_21.03.2014 | Host Interactions of HIV | 5.20 × 10⁻¹⁴ | 2.60 × 10⁻¹¹ |
| | factors | | |
| REACTOME_21.03.2014 | Disease | 1.09 × 10⁻¹³ | 5.42 × 10⁻¹¹ |
| REACTOME_21.03.2014 | Processing of Capped Intron- | 2.25 × 10⁻¹³ | 1.12 × 10⁻¹⁰ |
| | Containing Pre-mRNA | | |
| REACTOME_21.03.2014 | Late Phase of HIV Life Cycle | 1.33 × 10⁻¹² | 6.63 × 10⁻¹⁰ |
| REACTOME_21.03.2014 | Immune System | 1.10 × 10⁻¹¹ | 5.46 × 10⁻⁹ |
| REACTOME_21.03.2014 | Antiviral mechanism by IFN- | 7.60 × 10⁻¹¹ | 3.76 × 10⁻⁸ |
| | stimulated genes | | |
| REACTOME_21.03.2014 | ISG15 antiviral mechanism | 7.60 × 10⁻¹¹ | 3.76 × 10⁻⁸ |
| REACTOME_21.03.2014 | Nuclear Envelope Breakdown | 3.42 × 10⁻¹⁰ | 1.69 × 10⁻⁷ |
| REACTOME_21.03.2014 | mRNA Splicing - Minor | 6.57 × 10⁻¹⁰ | 3.24 × 10⁻⁷ |
| | Pathway | | |
| REACTOME_21.03.2014 | Latent infection of Homo | 1.08 × 10⁻⁹ | 5.31 × 10⁻⁷ |
| | sapiens with Mycobacterium | | |
| | tuberculosis | | |
| REACTOME_21.03.2014 | Phagosomal maturation (early | 1.08 × 10⁻⁹ | 5.31 × 10⁻⁷ |
| | endosomal stage) | | |
| REACTOME_21.03.2014 | Nuclear Pore Complex (NPC) | 4.37 × 10⁻⁹ | 2.15 × 10⁻⁶ |
| | Disassembly | | |
| REACTOME_21.03.2014 | Insulin receptor recycling | 5.06 × 10⁻⁹ | 2.48 × 10⁻⁶ |
| REACTOME_21.03.2014 | Transcriptional Regulation of | 5.44 × 10⁻⁹ | 2.66 × 10⁻⁶ |
| | White Adipocyte | | |
| | Differentiation | | |
| REACTOME_21.03.2014 | Activation of NF-kappaB in B | 7.31 × 10⁻⁹ | 3.57 × 10⁻⁶ |
| | Cells | | |
| REACTOME_21.03.2014 | Regulation of mRNA Stability | 7.74 × 10⁻⁹ | 3.77 × 10⁻⁶ |
| | by Proteins that Bind AU-rich | | |
| | Elements | | |
| REACTOME_21.03.2014 | Cytokine Signaling in Immune | 9.45 × 10⁻⁹ | 4.59 × 10⁻⁶ |
| | system | | |
| REACTOME_21.03.2014 | Transport of | 1.55 × 10⁻⁸ | 7.51 × 10⁻⁶ |
| | Ribonucleoproteins into the | | |
| | Host Nucleus | | |

### Network Centrality of Virus-Target Genes in the Human Protein Interaction Network

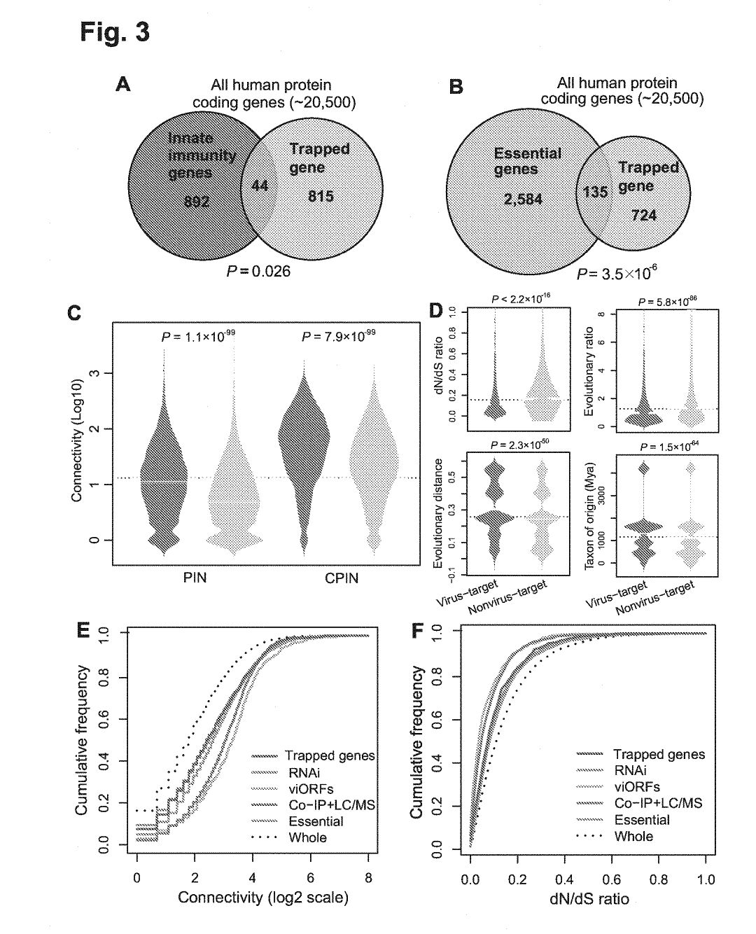

Of 859 host genes identified by gene-trap insertional mutagenesis, there was enrichment for genes associated with innate immunity (P=0.026, Fisher's exact test, FIG. 3A), suggesting that the identified host gene set may mediate immune responses (Pichlmair A, et al. (2012) Nature 487: 486-490). Essential genes, whose knockout result in lethality or infertility, are important for studying the robustness of a biological system (Chen W H, et al. (2012) Nucleic Acids Res 40: D901-906). Furthermore, there was also a significant enrichment for essential genes (P=3.5×10⁻⁶, FIG. 3B). To further investigate the biological functions of the identified virus-target genes, we examined topological network features, such as the degree of connectivity of virus-target gene products (proteins) in the human protein interactome. Considering that the current publicly-available human protein interaction databases are still incomplete, we constructed 5 different, yet complementary human protein interaction networks: a global physical protein interaction network (PIN), an atomic resolution three-dimensional structural protein interaction network (3DPIN), a kinase-substrate interaction network (KSIN), an innate immunity protein interaction network (INPIN), and a broad context computationally predicted protein interaction network (CPIN), based on previous studies (Cheng F, et al. (2014); Cheng F, et al. (2014)). FIG. 3C shows that the connectivity of virus-target proteins was significantly stronger than non-virus target proteins in the PIN (P=1.1×10⁻⁹⁹, Wilcoxon rank-sum test) and CPIN (P=7.9×10⁻¹¹⁰) datasets, respectively. In addition, we defined “hubs” as those nodes that ranked in the top 20% of the connectivity distribution, as done previously (Cheng F, et al. (2014); Cheng F, et al. (2014)). We found that virus-target proteins were significantly enriched in hubs in all 5 human protein interaction networks: PIN (P=1.7×10⁻⁷³, Fisher's exact test, Table 3), CPIN (P=8.4×10⁻⁷), INPIN (P=6.0×10⁻⁵), 3DPIN (P=7.4×10⁻⁴), and KSIN (P=6.2×10⁻¹⁰). Moreover, the virus-target proteins showed a tendency for greater enrichment of hubs in the INPIN dataset than in the CPIN dataset (P=6.6×10⁻³). The INPIN dataset contained a specific collection of PPIs involved in innate immunity-related pathways (Breuer K, et al. (2013) Nucleic Acids Res 41: D1228-1233), and our INPIN network analysis suggested that the immune system plays an important role during viral replication (FIG. 3A). In addition, we investigated the connectivity distribution of our 859 host genes with three published host gene sets. We found a comparable connectivity distribution of our 859 host genes with the RNAi gene set, although they were marginally lower in terms of significance than that observed with the viORFs and Co-PI+LC/MS gene sets (FIG. 3E). These observations suggest the reliability of gene-trap insertional mutagenesis, relative to results obtained using other technologies such as RNAi, Co-IP+LC/MS, and viORFs.

TABLE 3
Network topological (connectivity) analysis in
five independent protein interaction networks.

| | Number of Hub | Number of non-hub | |
|---|---|---|---|
| Network | proteins | proteins | Fisher's exact test |
| 3DPIN | 201 | 547 | 7.4 × 10⁻⁴ |
| INPIN | 171 | 580 | 6.0 × 10⁻⁵ |
| KSIN | 190 | 485 | 6.2 × 10⁻¹⁰ |
| PIN | 861 | 1,745 | 1.7 × 10⁻⁷³ |
| CPIN | 945 | 1,894 | 8.4 × 10⁻⁷ |

### Purifying Selection and Evolutionary Origins of Virus-Target Genes

To provide insight into the evolutionary factors underlying the selection of host genes used by viruses, we examined the selective pressure and evolutionary rates of the virus-target genes identified. We computed non-synonymous and synonymous substitution rate ratios (dN/dS ratios) using human-mouse orthologous gene pairs (See Methods). A dN/dS ratio of 1 signifies neutral evolution, a ratio of <1 indicates purifying selection, and a ratio of >1 indicates positive Darwinian selection. The boxplots in FIG. 3D show that virus-target genes tend to undergo purifying selection (i.e., the selective removal of alleles that are deleterious) in human protein evolutionary histories. Moreover, virus-target genes displayed stronger purifying selection (lower dN/dS ratios and evolutionary rate ratios) than did non-virus target genes (P<2.2×10⁻¹⁶, Wilcoxon rank-sum test), as shown in FIG. 3D. For example, several genes with the lowest dN/dS ratios (0) such as RAB1A (Pechenick Jowers T, et al. (2015) Virology 475: 66-73), PCBP1 (Zhou X, et al. (2012) Cell Res 22: 717-727), PCBP2 (You F, et al. (2009) Nat Immunol 10: 1300-1308), and ARF6 (Marchant D, et al. (2009) J Gen Virol 90: 854-862) were previously reported to be involved in viral replication or antiviral signaling pathways. However, only one gene (DEFB118), which was also implicated in viral replication-related pathway (Yenugu S, et al. (2004) Endocrinology 145: 3165-3173) had a dN/dS ratio large than 1 (1.1).

The evolutionary history of a protein sequence often reflects its functional evolution. We next investigated the evolutionary origin of virus-target gene products. The average time of divergence (1348.6±20.0 million years ago [mya]) for virus-target gene products was significantly longer than that of non-virus target gene products (1131.3±8.7 mya, P=2.3×10⁻⁵⁰; FIG. 3D). Furthermore, the average evolutionary distance of virus-target gene products was also significantly higher than that observed for non-virus target gene products (P=1.5×10⁻⁶⁴; FIG. 3D). We next compared the dN/dS ratio distribution for our 859 host genes with that of three published host gene sets. Compared with our set of 859 host genes, a similar trend was observed with the RNAi gene set (FIG. 3F).

### Regulating the Host Cell Cycle Program

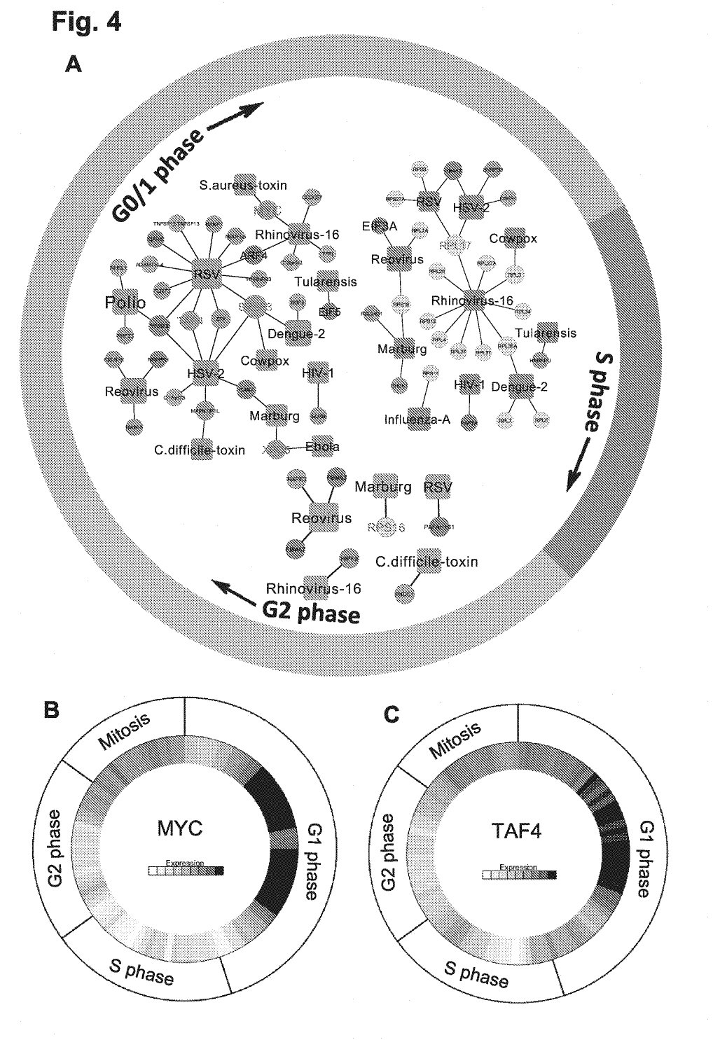

Most viruses are known to regulate host cell cycle program (Dyer M D, et al (2008) PLoS Pathog 4: e32; Taterka J, et al. (1994) J Clin Invest 94: 353-360). We assembled 986 human host cell cycle genes mediating G0/1, S, and G2 phases from a previous study (Kittler R, et al. (2007) Nat Cell Biol 9: 1401-1412). We found that the 859 host genes identified by gene-trap insertional mutagenesis were significantly enriched in terms of human cell cycle genes (P=6.5×10⁻⁵, Fisher's exact test). We next built a cell cycle phase-specific sub-network to systematically explore the cell cycle programing mechanisms for our host gene set (FIG. 4). G0/1 phase is the initial stage of the cell cycle. FIG. 4A shows that several genes important for viral replication also mediate progression though G0/G1 phase, including MYC, ARF4, SRSF3, TAF4, XPOS, and EIFS. ARF4 promotes enterovirus 71 replication (Wang J, et al. (2014) PLoS One 9: e99768), susceptibility to Chlamydia trachomatis and Shigella flexneri (Reiling J H, et al. (2013) Nat Cell Biol 15: 1473-1485), and dengue flavivirus secretion Kudelko M, et al. (2012) J Biol Chem 287: 767-777). Two previous studies indicated that TAF4 plays critical roles in herpes simplex virus type 1 infection (Quadt I, et al. (2006) Virus Res 115: 207-213) and transcriptional activation of Epstein-Barr virus (Yang Y C and Chang L K (2013) PLoS One 8: e54075). Here, we showed that TAF4 might mediate RSV and HSV-2 replication by regulating G0/1 phase using both gene-trap insertional mutagenesis and bioinformatics analysis. Furthermore, expression data analysis using Cyclobase (Santos A, et al. (2015) Nucleic Acids Res 43: D1140-1144) further confirmed that TAF4 regulates cell cycle in G1 phase, as shown in FIG. 4B. MYC, encoding c-Myc, is involved in the replication of multiple viruses, such as Epstein-Barr virus (Lacy J, et al. (1987) Proc Natl Acad Sci USA 84: 5838-5842; Fanidi A, et al. (1998) J Virol 72: 8392-8395). Here, we found that MYC may mediate rhinovirus-16 replication by regulating G0/G1 phase (FIGS. 4A and 4C). In addition to G0/1 phase, we also found that several genes (e.g. RPL17 and RPS16) regulate S or G2 phase transition, in addition to viral replication (FIG. 4A). RPL17, encoding 60S ribosomal protein L17 important for protein synthesis, plays critical roles in the replication of several viruses (Walsh D, et al. (2013) Cold Spring Harb Perspect Biol 5: a012351), including hepatitis C virus (Gupta R, et al. (2012) J Transl Med 10: 54), HSV-1 (Simonin D, et al. (1997) J Gen Virol 78 (Pt 2): 435-443), and potato virus A (Hafren A, et al. (2013) J Virol 87: 4302-4312). Collectively, these observations further suggested that the host cell cycle program plays important roles during viral replication by regulating specific cell cycle phases.

### Viral Perturbations of Cellular Networks Reflect Disease Etiology

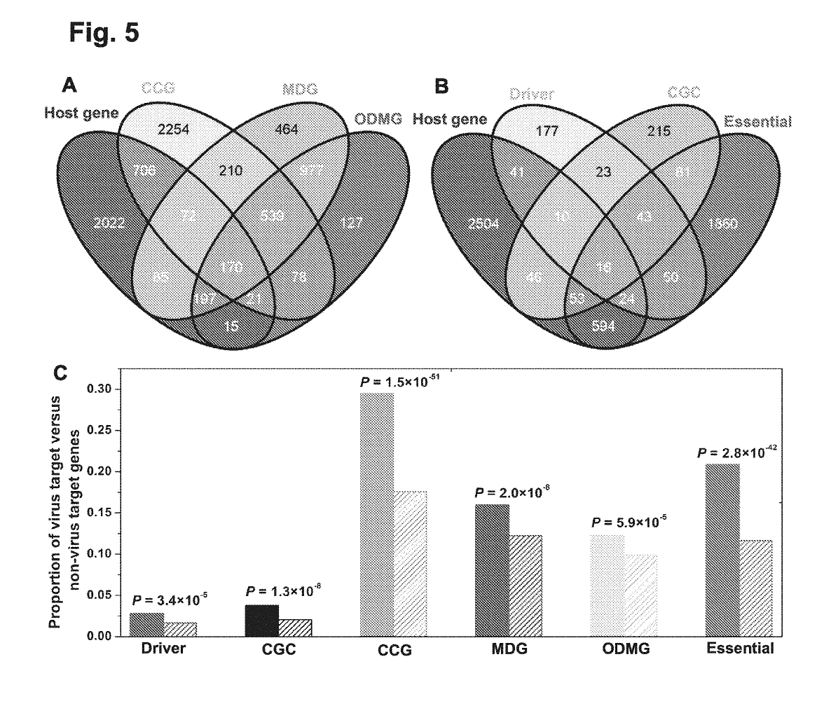

Understanding the interrelations between cellular host genes targeted by viral proteins and disease-susceptibility genes may reveal critical information for disease etiology Rozenblatt-Rosen 0, et al. (2012) Nature 487: 491-495; Gulbahce N, et al. (2012) PLoS Comput Biol 8: e1002531). We investigated the overlap between virus-target genes and the gene sets implicated in Mendelian diseases, orphan diseases, and cancer (FIGS. 5A and 5B). FIG. 5C shows that virus-target genes are significantly enriched in Mendelian disease genes (MDG; P=2.0×10⁻⁸), orphan disease-mutated genes (ODMG; P=5.9×10⁻⁵), and those in the catalogue of cancer genes (CCG P=1.5×10⁻⁵¹).

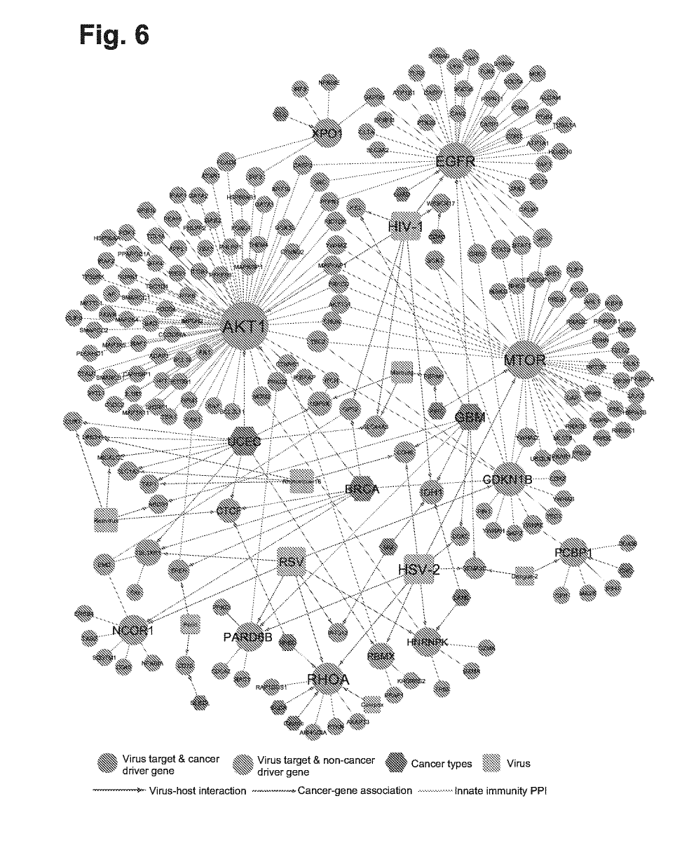

Data from a previous study showed that genomic variations and tumor viruses might cause cancer through related mechanisms (Rozenblatt-Rosen 0, et al. (2012)). Thus, we examined how virus-target genes promote tumorigenesis or are involved in cancer etiology. We selected 384 genes that are significantly mutated in cancer (cancer-driver genes) from several large-scale cancer genome projects. Interestingly, a significant association (P=3.4×10⁻⁵) was observed between the cancer-related genes and genes implicated in viral infection identified by our gene-trap studies and prior RNAi screens. As shown in FIG. 6 and Table 4, 26 of the 384 cancer driver genes were identified in gene-trap studies with lytic viruses (such as CTCF, RHOA, CDKN1B, and CUX1), while 66 of the 384 genes were previously identified in RNAi screens (such as PIK3CA, HRAS, EGFR, AKT1, and IDH1).

TABLE 4
Experimental evidences from literature data to support the relationship between
influenza virus infection (25 influenza virus infection-related genes identified by our gene
trapped insertional mutagenesis) and cancers.

| | | | | Interaction |
|---|---|---|---|---|
| Gene | | | Influenza-A | with |
| symbol | Gene name | Cancer-related | related | virus proteins |
| AHDC1 | AT hook, DNA binding motif, | NA | NA | NA |
| | containing 1 | | | |
| AMT | aminomethyltransferase | NA | NA | NA |
| ASPSCR1 | alveolar soft part sarcoma | Alveolar soft | NA | NA |
| | chromosome region, candidate 1 | part sarcoma [9] | | |
| BCL6 | B-cell CLL/lymphoma 6 | Diffuse large B | NA | [12] |
| | | cell lymphoma | | |
| | | [10, 11] | | |
| BSN | bassoon presynaptic | NA | NA | NA |
| | cytomatrix protein | | | |
| CAV2 | caveolin 2 | Multiple cancer | NA | [14] |
| | | types [13] | | |
| CDH23 | cadherin-related 23 | [15] | [15] | [15] |
| CEP170 | centrosomal protein 170 kDa | [16] | [15] | [15] |
| DAG1 | dystroglycan 1 (dystrophin- | NA | NA | NA |
| | associated glycoprotein 1) | | | |
| EEF1A1 | eukaryotic translation | Prostate | [18, 19] | [18] |
| | elongation factor 1 alpha 1 | cancer [17] | | |
| FLT3LG | fms-related tyrosine kinase 3 | NA | NA | NA |
| | ligand | | | |
| GPAM | glycerol-3-phosphate | Breast | NA | NA |
| | acyltransferase, mitochondrial | cancer [20] | | |
| HAS2 | hyaluronan synthase 2 | Breast | NA | NA |
| | | cancer [21] | | |
| IRS1 | insulin receptor substrate 1 | Multiple | NA | [22] |
| | | cancers [22, 23] | | |
| RSL24D1P4 | ribosomal L24 domain | NA | NA | NA |
| (pseudo) | containing 1 pseudogene 4 | | | |
| LPP | LIM domain containing | Breast | NA | NA |
| | preferred translocation partner | cancer [24] | | |
| | in lipoma | | | |
| MLLT10 | myeloid/lymphoid or mixed- | Brain cancer [25] | NA | NA |
| | lineage leukemia (trithorax | | | |
| | homolog, Drosophila); | | | |
| | translocated to, 10 | | | |
| MYOF | myoferlin | Breast | NA | NA |
| | | cancer [26] | | |
| NME2P1 | NME/NM23 nucleoside | NA | NA | NA |
| (Pseudo) | diphosphate kinase 2 | | | |
| | pseudogene 1 | | | |
| PCDH9 | protocadherin 9 | Glioma [27] | [28] | [28] |
| PLD5 | phospholipase D family, | NA | NA | NA |
| | member 5 | | | |
| PTPN1 | protein tyrosine phosphatase, | Breast | NA | [30] |
| | non-receptor type 1 | cancer [29] | | |
| PXN | paxillin | Multiple | | [33] |
| | | cancers [31, 32] | | |
| RCC1 | regulator of chromosome | Cancers [34] | [19, 35] | [36, 37] |
| | condensation 1 | | | |
| RNU105A | RNA, U105A small nucleolar | NA | NA | NA |
| (snoRNA) | | | | |
| RPS11 | ribosomal protein S11 | Colon | [19] | [19] |
| | | cancer [38] | | |
| SH3GL2 | SH3-domain GRB2-like 2 | Multiple | NA | [41] |
| | | cancers [39, 40] | | |
| SNORA73A | small nucleolar RNA, H/ACA | NA | NA | NA |
| (snoRNA) | box 73A | | | |
| SNORA73B | small nucleolar RNA, H/ACA | NA | NA | NA |
| (snoRNA) | box 73B | | | |
| SNORD35B | small nucleolar RNA, C/D | NA | NA | NA |
| (snoRNA) | box 35B | | | |
| SPATA19 | spermatogenesis associated 19 | Prostate cancer | NA | NA |
| | | [42] | | |
| TCTA | T-cell leukemia translocation | NA | NA | NA |
| | altered | | | |
| TECTB | tectorin beta | NA | NA | NA |
| TES | testis derived transcript (3 | NA | NA | NA |
| | LIM domains) | | | |
| UBE2E3 | ubiquitin-conjugating enzyme | NA | NA | NA |
| | E2E 3 | | | |
| WASF2 | WAS protein family, member 2 | Gastric cancer | NA | [44] |
| | | [43] | | |

The human CTCF gene encodes the CTCF transcriptional repressor, which mediates transcriptional regulation, insulator activity, and the regulation of chromatin architecture (Rubio E D, et al. (2008) Proc Natl Acad Sci USA 105: 8309-8314). Data from several recent cancer genome projects showed that CTCF mutations are significantly associated with breast cancer (Network TCGA (2012) Nature 490: 61-70), head and neck cancer (Stransky N, et al. (2011) Science 333: 1157-1160), and uterine cancer (N, Kandoth et al. (2013) Nature 497: 67-73). Interestingly, CTCF is involved in reovirus replication (FIG. 6). Pre-clinical studies indicate that treatment with reovirus is associated with significant anticancer activity in various cancer types (Kelly K, et al. (2009) Expert Opin Biol Ther 9: 817-830), such as breast cancer cell lines and cancer stem cells (Marcato P, et al. (2009) Mol Ther 17: 972-979; Norman K L, et al. (2002) Hum Gene Ther 13: 641-652), ovarian cancer (Hirasawa K, et al. (2002) Cancer Res 62: 1696-1701), colon cancer (Hirasawa K, et al. (2002)), and head and neck cancer (Twigger K, et al. (2012) BMC Cancer 12: 368). Furthermore, a recent study showed that infection with an oncolytic adenovirus (Ad315-E1A) or a replication-deficient recombinant adenovirus (Ad315-EGFP) significantly decreased cell viability and induced apoptosis of colon cancer cells in vitro and reduced tumor growth in a xenograft model by targeting CTCF binding sites (CCCTC) [57]. Thus, developing a novel reovirus that targets CTCF transcription factor binding sites by potential partial inhibition of viral replication or partial oncolytic activity may provide a potential strategy for targeted cancer therapy. Isocitrate dehydrogenase 1 (encoded by IDH1) regulates cancer cell metabolism and is frequently mutated in multiple cancer types, such as glioblastoma (Brennan C W, et al. (2013) Cell 155: 462-477), acute myeloid leukemia (Network TCGAR (2013) N Engl J Med 368: 2059-2074), and multiple myeloma (Lawrence M S, et al. (2014) Nature 505: 495-501) (FIG. 6). Previous epidemiological studies demonstrated the coincidence of HIV infection and glioblastoma (Hall J R and Short S C (2009) Clin Oncol (R Coll Radiol) 21: 591-597), acute myeloid leukemia (Aboulafia D M, et al. (2002) Acute myeloid leukemia in patients infected with HIV-1. AIDS 16: 865-876), and multiple myeloma (Yee T T, et al. (2001) Am J Hematol 66: 123-125). Thus, the human IDH1 gene product interacting with HIV-1 may represent a potential etiological mechanism of tumorigenesis for these cancer types during viral replication (FIG. 6).

It has been previously reported that influenza viruses rely on the PI3K-Akt-mTOR axis for successful replication, as well as on the activity of cell cycle regulators, which are often dysregulated in cancer (Shaw M L (2011) Rev Med Virol 21: 358-369). To explore the relationship between influenza viral replication and cancer, we systematically investigated the 25 host genes implicated in influenza virus replication identified by gene-trap insertional mutagenesis. As shown in Table 4, among 25 host genes, 5 genes (CEP170 (York A, et al. (2014) J Virol 88: 13284-13299), EEF1A1 (Karlas A, et al. (2010) Nature 463: 818-822), PCDH9 (Marazzi I, et al. (2012) Nature 483: 428-433), RCC1, and RPS11 (Watanabe T, et al. (2014))) were previously reported to be involved in influenza-A replication. Interestingly, we found that several influenza replication-related genes EEF1A1 (Scaggiante B, et al. (2012) Br J Cancer 106: 166-173), IRS1 (Reiss K, et al. (2012) J Cell Physiol 227: 2992-3000), RPS11 (Lai M D and Xu J (2007) Curr Genomics 8: 43-49), PCDH9 (Wang C, et al. (2014) J Mol Neurosci 52: 250-260), and SPATA19 (Ghafouri-Fard S, et al. (2010) Arch Med Res 41: 195-200) were involved in tumorigenesis. For example, FIG. 4A shows that RPS11 may mediate influenza-A replication by regulating S phase, using gene-trap insertional mutagenesis and cell cycle phase-specific sub-network analysis. Lai and Xu found that RPS11 might play important roles in colorectal cancer (Lai M D and Xu J (2007)). Thus, host genes mediating viral replication (e.g. influenza A) often drive cancer through shared crosstalk pathways (e.g., cell cycle) (Ilkow C S, et al. (2015) Nat Med 21: 530-536). Collectively, the virus-target genes identified were significantly enriched for cancer-related genes, suggesting that virus-host perturbation networks may shed valuable insight for prioritizing disease-associated or cancer-driver mutations (Molyneux S D, et al. (2014) Nat Genet 46: 964-972).

### Identifying New Antiviral Targets and Indications for Existing Drugs

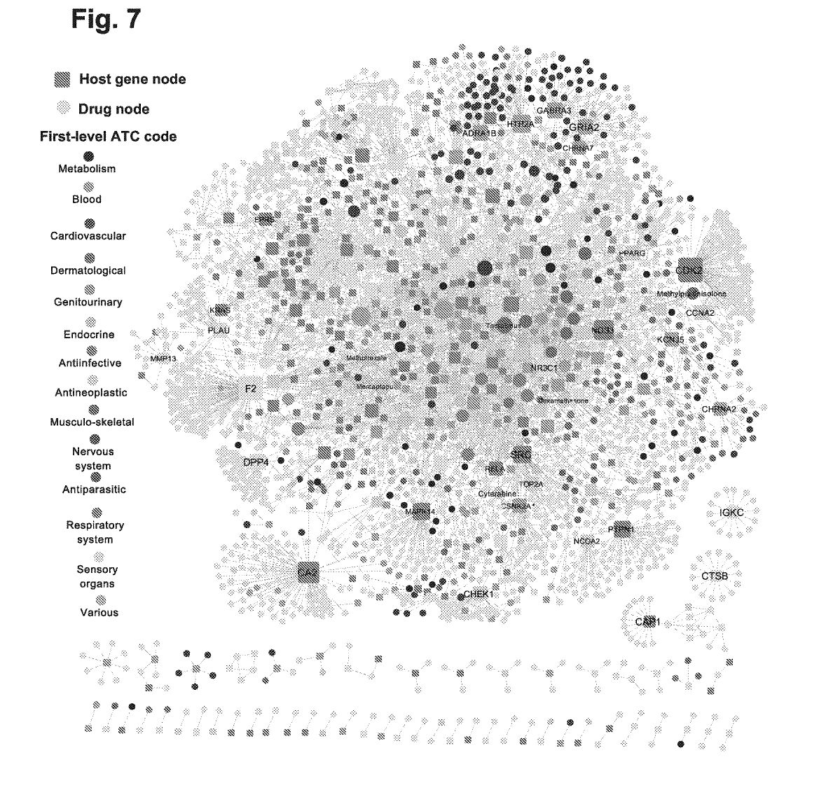

To identify new druggable targets for antiviral pharmacotherapy, we cross-referenced all virus target genes identified by previous global RNAi screens and gene-trap insertional mutagenesis studies with 3 drug-target databases, namely the DrugBank (Wishart D S, et al. (2008) Nucleic Acids Res 36: D901-906), the Therapeutics Target Database (Zhu F, et al. (2012) Nucleic Acids Res 40: D1128-1136), and PharmGKB (Hernandez-Boussard T, et al. (2008) Nucleic Acids Res 36: D913-918). In total, we found 691 virus target genes (138 host genes identified by gene-trap insertional mutagenesis) whose products can be targeted by approved drugs, investigational drugs, or pre-clinical agents, which are referred to here as “druggable virus-target genes.” We performed KEGG pathway analysis for these 691 druggable virus-target genes using GlueGO. The most significantly enriched pathways included Epstein-Barr virus infection (q=7.0×10⁻¹³), osteoclast differentiation (q=3.4×10⁻⁷), proteasome (q=1.9×10⁻⁷), the neurotrophin signaling pathway (q=1.1×10⁻⁶), ERBB signaling pathway (q=2.0×10⁻⁶), influenza-A (q=1.0×10⁻⁵), T cell receptor signaling pathways (q=1.3×10⁻⁵), and the MAPK signaling pathway (q=5.7×10⁻⁵, Table 5). FIG. 7 showed a bipartite drug-target interaction network connecting 691 virus-target genes (squares) and 2,071 existing drugs (circles). All drugs were grouped using the anatomical therapeutic chemical classification system. Multiple drugs exist for several gene products, including CDK2, NOS3, NR3C1, MAPK14, SRC, and CHEK1 (FIG. 7), providing new opportunities for targeting those genes for antiviral pharmacotherapy. Interestingly, most cancer drugs often target host genes mediating viral replication. KEGG pathway enrichment analysis showed that several of the most significant pathways are involved in cancer (Table 5), such as chronic myeloid leukemia (q=5.3×10⁻¹⁰), pathways in cancer (q=1.9×10⁻⁸), prostate cancer (q=2.3×10⁻⁸), and pancreatic cancer (q=4.8×10⁻⁸). Among the 2,071 drugs shown in FIG. 7, 52 drugs are known anti-infective drugs, such as lamivudine, ritonavir, ribavirin, zidovudine, and rifampin. FIG. 7 thus provides useful information for repurposing approved therapeutic agents as novel antiviral indications.

TABLE 5
Top 20 significantly enriched KEGG pathways for
691 druggable host genes identified in previous RNAi screens
and our gene-trap insertional mutagenesis studies.

| | | | Adjust |
|---|---|---|---|
| | | | P value |
| | | Term | (Bonferroni |
| Pathway Source | GO Term | P value | correction) |
| KEGG_24.05.2012 | Epstein-Barr virus | 3.87 × 10⁻¹⁵ | 7.00 × 10⁻¹³ |
| | infection | | |
| KEGG_24.05.2012 | Chronic myeloid | 2.93 × 10⁻¹² | 5.27 × 10⁻¹⁰ |
| | leukemia | | |
| KEGG_24.05.2012 | Pathways in cancer | 1.05 × 10⁻¹⁰ | 1.88 × 10⁻⁷ |
| KEGG_24.05.2012 | Prostate cancer | 1.27 × 10⁻¹⁰ | 2.26 × 10⁻⁷ |
| KEGG_24.05.2012 | Pancreatic cancer | 2.69 × 10⁻¹⁰ | 4.76 × 10⁻⁷ |
| KEGG_24.05.2012 | Proteasome | 9.09 × 10⁻¹⁰ | 1.60 × 10⁻⁷ |
| KEGG_24.05.2012 | Osteoclast | 1.54 × 10⁻⁹ | 2.69 × 10⁻⁷ |
| | differentiation | | |
| KEGG_24.05.2012 | Neurotrophin signaling | 4.95 × 10⁻⁹ | 8.62 × 10⁻⁷ |
| | pathway | | |
| KEGG_24.05.2012 | ErbB signaling | 9.39 × 10⁻⁹ | 1.62 × 10⁻⁶ |
| | pathway | | |
| KEGG_24.05.2012 | Influenza A | 5.94 × 10⁻⁸ | 1.02 × 10⁻⁵ |
| KEGG_24.05.2012 | T cell receptor | 7.82 × 10⁻⁸ | 1.34 × 10⁻⁵ |
| | signaling pathway | | |
| KEGG_24.05.2012 | Glioma | 2.91 × 10⁻⁷ | 4.95 × 10⁻⁵ |
| KEGG_24.05.2012 | MAPK signaling | 3.34 × 10⁻⁷ | 5.65 × 10⁻⁵ |
| | pathway | | |
| KEGG_24.05.2012 | Insulin signaling | 5.27 × 10⁻⁷ | 8.86 × 10⁻⁵ |
| | pathway | | |
| KEGG_24.05.2012 | Acute myeloid | 7.47 × 10⁻⁷ | 1.25 × 10⁻⁴ |
| | leukemia | | |
| KEGG_24.05.2012 | Small cell lung cancer | 7.91 × 10⁻⁷ | 1.31 × 10⁻⁴ |
| KEGG_24.05.2012 | Bladder cancer | 1.08 × 10⁻⁶ | 1.78 × 10⁻⁴ |
| KEGG_24.05.2012 | Renal cell carcinoma | 1.10 × 10⁻⁶ | 1.80 × 10⁻⁴ |
| KEGG_24.05.2012 | Non-small cell lung | 1.60 × 10⁻⁶ | 2.60 × 10⁻⁴ |
| | cancer | | |
| KEGG_24.05.2012 | Chemokine signaling | 2.11 × 10⁻⁶ | 3.42 × 10⁻⁴ |
| | pathway | | |

Naturally, drugs targeting viral proteins tend to be virus-specific. Drugs directed against cellular proteins or signaling pathways potentially have a much broader spectrum of antiviral activities, as the replication of different viruses often depends on similar cellular mechanisms. In this study, we developed a computational approach to identify novel antiviral indications for existing drugs by incorporating drug-gene signatures from the CMap into the global virus-host interactome identified by previous global RNAi screens and gene-trap insertional mutagenesis studies (FIG. 1F). The hypothesis underlying this computational approach is that if a given drug up- or down-regulates cellular genes to then inhibit the replication of a specific virus, then this drug may be potentially useful as anti-infective (FIG. 1F). We calculated P values by Fisher's exact test and corrected P values using Bonferroni's correction for each drug-virus pair, with q<0.1 set as the cutoff for identifying significant drug-virus pairs for antiviral drug repositioning. Using this cutoff, we found 213 significant drug-virus pairs connecting 171 drugs and 29 viruses. Recently, He et al. screened ˜3,800 small molecules for the treatment of hepatitis C virus (HCV) infection and identified 39 chlorcyclizine analogs with 50% maximal effective concentration (EC₅₀) less than 100 μM using a cell-based quantitative high-throughput screening platform (He S, et al. (2015) Sci Transl Med 7: 282ra249). Here, we computationally repurposed 11 potential drugs for anti-HCV infection with q<0.1. Among 11 significant candidates, the drug homochlorcyclizine (7^{th}-most significant prediction, q=0.046) was previously reported to have high anti-HCV activity with an EC₅₀ value of 0.47 μM (He S, et al. (2015)). In addition, among the top 90 predicted drugs, three hits, including homochlorcyclizine (EC₅₀=0.47 μM), clemizole (EC₅₀=7.15 μM), and orphenadrine (EC₅₀=10.5 μM) were previously validated, suggesting higher enrichment (odds ratio=3.3, P=0.07; Fisher's exact test) occurred in our computational approach, compared to traditional experimental screens (He S, et al. (2015)). We next evaluated 2 case studies to discover new anti-HIV-1 and anti-Ebola indications for existing drugs.

### Identifying New Anti-HIV-1 Indications for Existing Drugs

Our bioinformatics analyses identified 16 drugs that have potential anti-HIV-1 indications (q<0.1). Alsterpaullone, a small molecular cyclin-dependent kinase inhibitor, regulates cell cycle progression. Here, alsterpaullone was significantly predicted to have an anti-HIV-1 indication (q=0.011). Recently, Guendel et al. found that alsterpaullone is a potent inhibitor of HIV-1, with an approximate IC₅₀ value of 150 nM (Guendel I, (2010) AIDS Res Ther 7: 7). Lycorine, a toxic crystalline alkaloid, inhibits protein synthesis and ascorbic acid biosynthesis. In this study, lycorine was predicted to have anti-HIV-1 activity, with the fourth-lowest adjusted P value observed (q=0.014, Table 6). Virjsen et al. found that alkaloid lycorine inhibits viral protein synthesis in poliovirus-infected HeLa cells (Vrijsen R, (1986) J Biol Chem 261) and Liu et al. found that lycorine reduces mortality of human enterovirus 71-infected mice by inhibiting viral replication (Liu J, et al. (2011) Virol J 8: 483). Moreover, the amary-llidaceae alkaloid lycorine isolated from the bulbs of Leucojum vernum possesses anti-HIV-1 activity in MT4 cells with an IC₅₀ value of 0.4 μg/mL (Szlavik L, et al. (2004) Planta Med 70: 871-873). Sanguinarine, a toxic quaternary ammonium salt, was predicted to have an anti-HIV-1 indication, with the fifth lowest q value (q=0.019). Tan et al. found that sanguinarine nitrate shows moderate inhibitory activity, with an IC₅₀ of 50-150 μg/mL against the HIV-1 reverse transcriptase (Tan G T, et al. (1991) J Nat Prod 54: 143-154). Thus, among the top 5 predicted candidates, 4 agents have been validated in previous studies (Table 6), indicating the possibility that other top candidates have anti-HIV efficacy as well. In addition, we systemically searched top 20 predicted agents for potential anti-HIV indications. Table 6 shows that 6 additional agents have demonstrated experimental anti-HIV activity data, including fursultiamine (q=0.055) (Kv L N and Nguyen L T (2013) Int J Infect Dis 17: e221-227), trichostatin A (q=0.068) (Kiernan R E, et al. (1999) EMBO J 18: 6106-6118), doxorubicin (q=0.071) (Johansson S, et al. (2006) AIDS 20: 1911-1915), promethazine (q=0.081) (Lu W, et al. (2001) J Immunol 167: 2929-2935), 8-azaguanine (17^{th} highest significance, q=0.103) (Wong R W, et al. (2013) Nucleic Acids Res 41: 9471-9483), and staurosporine (20^{th} highest significance, q=0.145) (Aranda-Anzaldo A, Viza D (1992) FEBS Lett 308: 170-174), revealing a 50% success rate in computational prediction for the top 20 candidates. Taken together, these data suggest potential application of our method in identifying anti-HIV-1 indications for existing drugs as well.

TABLE 6
Anti-HIV activities of top 20 predicted candidates based on
the evidence from literatures.

| | | Adjusted p- | Refs of Anti- | |
|---|---|---|---|---|
| Predicted drug names | p-value | value | HIV activity | Ranks |
| camptothecin | 0.0000609 | 0.003715 | [45, 46] | Top 1 |
| sertaconazole | 0.0000939 | 0.005728 | NA | Top 2 |
| alsterpaullone | 0.0001745 | 0.010642 | [47] | Top 3 |
| lycorine | 0.0002327 | 0.014195 | [48] | Top 4 |
| sanguinarine | 0.000319 | 0.01946 | [49] | Top 5 |
| testosterone | 0.0006199 | 0.037815 | NA | Top 6 |
| amylocaine | 0.000741 | 0.045201 | NA | Top 7 |
| 2,6- | 0.0007523 | 0.04589 | NA | Top 8 |
| dimethylpiperidine | | | | |
| triprolidine | 0.0009069 | 0.055322 | NA | Top 9 |
| fursultiamine | 0.0009652 | 0.058877 | [50] | Top 10 |
| trichostatin A | 0.0011253 | 0.068646 | [51] | Top 11 |
| doxorubicin | 0.0011878 | 0.071269 | [52] | Top 12 |
| cobalt chloride | 0.0012114 | 0.073893 | NA | Top 13 |
| fulvestrant | 0.0012775 | 0.07793 | NA | Top 14 |
| promethazine | 0.0013481 | 0.080886 | [53, 54] | Top 15 |
| viomycin | 0.0013504 | 0.082375 | NA | Top 6 |
| 8-azaguanine | 0.0016932 | 0.103283 | [55] | Top 17 |
| 0179445-0000 | 0.0019612 | 0.119631 | NA | Top 18 |
| chlorphenesin | 0.0022216 | 0.135518 | NA | Top 19 |
| staurosporine | 0.0023821 | 0.145307 | [56, 57] | Top 20 |

### Identifying New Anti-Ebola Virus Indications for Existing Drugs

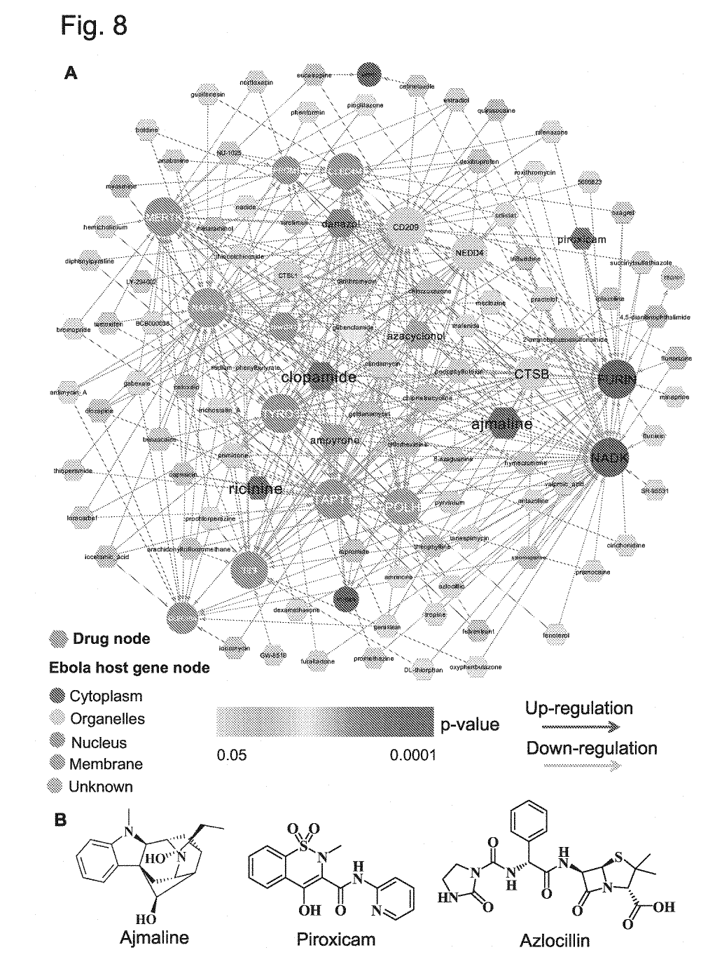

Infection by filoviruses such as the Ebola or Marburg viruses rapidly causes fatal hemorrhagic fever in humans, for which no approved antiviral agents are available (Strauss S (2014) Nat Biotechnol 32: 849-850). Thus, there is an urgent need to develop novel anti-Ebola virus agents, especially small molecule inhibitors. In total, 7 agents were predicted to have potential anti-Ebola indications, with q<0.1. The top 5 agents identified were ajmaline (q=0.002), ricinine (q=0.008), clopamide (q=0.016), piroxicam (q=0.029), and danazol (q=0.053). Ajmaline, an approved antiarrhythmic alkaloid, was predicted to have the most significant anti-Ebola indication (q=0.002, S9 Table). FIG. 8 revealed that ajmaline up-regulates expression of several important Ebola-related genes, such as MERTK, FURIN, TYRO3, FURIN, and CTSB. Several previous studies showed that MERTK (Shimojima M, et al. (2006) J Virol 80: 10109-10116) and FURIN (Volchkov V E, et al. (1998) Proc Natl Acad Sci USA 95: 5762-5767) play important roles in mediating the cellular entry of Ebola and processing of the Ebola virus glycoprotein. Shimojima et al. demonstrated that TYRO3 mediates the entry of Ebola and Marburg viruses (Shimojima M, et al. (2006)). Volchkov et al. found that proteolytic processing of the envelope glycoprotein by FURIN may play an important role in Ebola virus pathogenicity (Volchkov V E, et al. (1998)). Chandran et al. reported that Ebola virus entry depends on CTSB expression (Chandran K, et al. (2005) Science 308: 1643-1645). Recently, a bis-benzylisoquinoline alkaloid, tetrandrine, was found to inhibit entry of Ebola virus into host cells in vitro and preliminary studies in mice further confirmed the therapeutic efficacy against Ebola by inhibiting two pore calcium channel protein (Sakurai Y, et al. (2015) Science 347: 995-998). Moreover, a previous study showed that ajmaline (≦20 μM) exerted comparable pharmacological activity compared with tetrandrine (5-10 μM) by inhibiting calcium channel protein activity (Fish J M and Antzelevitch C (2004) Heart Rhythm 1: 210-217). Taken together, targeting MERTK, CTSB, TYRO3, and FURIN by alkaloid ajmaline may provide a novel therapeutic strategy against Ebola virus. Piroxicam is a non-steroidal anti-inflammatory drug that is used as a pain reliever. FIG. 8 shows that piroxicam up-regulates FURIN and MERTK expression, according to the CMap data (Lamb J, et al. (2006)). Thus, Azlocillin, an acylampicillin antibiotic, has an extended spectrum of antibacterial activity, and was also predicted here to have anti-Ebola virus activity (P=0.006). FIG. 8 shows that azlocillin up-regulates NADK and POLH expression and down-regulates TAPT1 and TYRO3 expression. Taken together, targeting MERTK, CTSB, TYRO3, and FURIN by existing agents (e.g. ajmaline) may provide potential strategies for Ebola virus prevention and therapy. Further study will be needed for to provide experimental validations, which we hope will be prompted by the findings herein.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of identifying a promising cellular antiviral or bacterial toxin drug target, comprising:
1) providing a plurality of potential antiviral or bacterial toxin drug targets;
2) generating an interactome including the potential drug targets using a systems-biology computational method; and
3) analyzing the interactome to identify one or more promising antiviral or bacterial toxin drug targets.

2. The method of claim 1, wherein the potential drug targets are identified by insertional mutation using a gene trapping vector.

3. The method of claim 1, wherein the potential drug targets are identified by insertional mutation using siRNA.

4. The method of claim 1, wherein the drug target is an antiviral drug target.

5. The method of claim 1, wherein the antiviral drug target is a target suitable for treatment of viral infection by a virus selected from the group consisting of bovine viral diarrhea virus, cowpox virus, Dengue fever virus, Ebola virus, HIV-1, Herpes Simplex virus, Marburg virus, poliovirus, reovirus, rhinovirus 2, rhinovirus 16, and respiratory syncytial virus.

6. The method of claim 1, wherein the drug target is a bacterial toxin drug target.

7. The method of claim 6, wherein the bacterial toxin drug target is a target suitable for decreasing toxicity by a bacterial toxin selected from the group consisting of the bacterial toxin is selected from the group consisting of Clostridium difficile TcdB toxin, C. perfringens α and β toxin, Helicobacter pylori vacuolating toxin, ricin toxin, and Staphylococcus aureus α toxin.

8. The method of claim 1, wherein the systems-biology computational method comprises a network analysis.

9. The method of claim 1, wherein the systems-biology computational method comprises a bioinformatics analysis.

10. The method of claim 1, wherein the systems-biology computational method comprises a diseasome enrichment analysis.

11. The method of claim 1, wherein the systems-biology computational method comprises an evolutionary feature analysis.

12. The method of claim 1, wherein the potential antiviral or bacterial toxin drug targets comprise one or more cancer-related genes or cancer-related gene expression products.

13. A method of identifying a new antiviral drug indication, comprising:
1) providing a plurality of potential antiviral drug targets;
2) generating an interactome including the potential antiviral drug targets using a systems-biology computational method;
3) analyzing the interactome to identify one or more promising antiviral drug targets; and
4) comparing a list of antiviral drug-gene signatures with the one or more promising antiviral drug targets to identify a new antiviral drug indication.

14. The method of claim 13, wherein the potential drug targets are identified by insertional mutation using a gene trapping vector.

15. The method of claim 13, wherein the potential drug targets are identified by insertional mutation using siRNA.

16. The method of claim 13, wherein the antiviral drug target is a target suitable for treatment of viral infection by a virus selected from the group consisting of bovine viral diarrhea virus, cowpox virus, Dengue fever virus, Ebola virus, HIV-1, Herpes Simplex virus, Marburg virus, poliovirus, reovirus, rhinovirus 2, rhinovirus 16, and respiratory syncytial virus.

17. A method of treating a subject having an HIV-1 infection by administering a therapeutically effective amount of a compound selected from the group consisting of alsterpaullone, lycorine, sanguinarine, testosterone, amylocaine, 2,6-dimethylpiperidine, triprolidine, fursultiamine, trichostatin A, and doxorubicin.

18. The method of claim 17, wherein the compound is alsterpaullone, lycorine, or sanguinarine.

19. A method of treating a subject having a RSV infection by administering a therapeutically effective amount of a compound selected from the group consisting of etamsylate, nicardipine, disulfiram, scoulerine, midecamycin, tyrphostin AG-825, hydroxyachillin, decamethonium bromide, PNU-0293363, and propantheline bromide.

20. A method of treating a subject having an HSV-2 infection by administering a therapeutically effective amount of a compound selected from the group consisting of meclofenoxate, nocodazole, ellipticine, nilutamide, thioridazine, calycanthine, PF-00562151-00, trichostatin A, valproic acid, and digitoxigenin.

21. A method of treating a subject having an Ebola virus infection by administering a therapeutically effective amount of a compound selected from the group consisting of piroxicam, azlocillin, and staurosporine.
